(19) United States
(12) Patent Application Publication
(10) Pub. No.: US 2014/0228422 A1
(43) Pub. Date: Aug. 14, 2014

(54) TARGETS FOR TREATMENT OF ER STRESS

(71) Applicants: Gökhan S. Hotamisligil, Wellesley, MA (US); Suneng Fu, Chestnut Hill, MA (US)

(72) Inventors: Gökhan S. Hotamisligil, Wellesley, MA (US); Suneng Fu, Chestnut Hill, MA (US)

(73) Assignee: PRESIDENT AND FELLOWS OF HARVARD COLLEGE, Cambridge, MA (US)

(21) Appl. No.: 14/029,890

(22) Filed: Sep. 18, 2013

Related U.S. Application Data
(63) Continuation of application No. PCT/US2012/029342, filed on Mar. 16, 2012.
(60) Provisional application No. 61/454,099, filed on Mar. 18, 2011.

(51) Int. Cl. C12N 15/113

(52) U.S. Cl. CPC C12N 15/1137
USPC 514/44 A

(57) ABSTRACT

The embodiments of the invention provide for genetic, chemical or dietary interventions that modulate hepatic phospholipid synthesis and/or endoplasmic reticulum (ER) calcium homeostasis function. More specifically, the present invention addresses modulation of the lipid composition of the hepatic stressed ER and/or improvement of the hepatic ER calcium metabolism to reduce ER stress and thus treat type 2 diabetes, fatty liver disease, atherosclerosis, inflammation, and/or dislipidemia.

### CROSS-REFERENCE TO RELATED APPLICATIONS

This application is a continuation of International Application No. PCT/US2012/029342 filed on Mar. 16, 2012, which designates the U.S., and which claims benefit under 35 U.S.C. §119(e) of U.S. Provisional Application No. 61/454,099 filed Mar. 18, 2011, the contents of each of which are incorporated herein by reference in their entireties.

### FEDERAL FUNDING

This invention was made with government support under Grants T32 ES7155-24, DK52539 and 1RC4-DK090942, awarded by the National Institutes of Health. The U.S. government has certain rights in the invention.

### FIELD

The present invention relates to molecular biology and cell metabolism.

### BACKGROUND

In recent years, the world has seen an alarming increase in metabolic diseases including obesity, insulin resistance, diabetes, fatty liver disease, and atherosclerosis. For example, over twenty million children and adults in the U.S., or 8% of the population, suffer from diabetes. Atherosclerosis is a leading cause of coronary heart disease and stroke, killing more than 600,000 Americans annually: more than 25% of all deaths in the U.S.

### SUMMARY

The embodiments of the invention provide for genetic, chemical or dietary interventions that modulate hepatic phospholipid synthesis and/or endoplasmic reticulum (ER) calcium homeostasis function. More specifically, the present invention addresses modulation of the lipid composition of the hepatic stressed ER and/or improvement of the hepatic ER calcium metabolism to reduce ER stress and thus treat type 2 diabetes, fatty liver disease, atherosclerosis, inflammation, and/or dislipidemia.

An embodiment provides for the overall modulation of cellular phospholipid synthesis, in particular correcting the abnormal distribution of PC and PE in the ER and other cell membranes and organelles, to modulate cellular functions and inflammation. For example, the PC/PE ratio is increased in the ER but decreased in the plasma membrane of obese subjects, therefore there is a clear imbalance regarding phospholipid distribution across different cellular compartments. Modulating the PC/PE ratio balance between cellular organelles is beneficial both on cellular level as well as the body level.

Another embodiment of the present invention provides for inhibitors of Pemt expression or PEMT activity, comprising genetic, molecular (e.g., drug) and/or specific dietary regimens, that modulate phospholipid synthesis in the ER, and thus regulate calcium homeostasis, glucose homeostasis, and insulin sensitivity. More specifically, down-modulation of hepatic PEMT lowers the hepatic PC/PE ratio from a higher ratio to the lower ratio observed in normal (e.g., non-obese, non-ER stressed) hepatic ER.

Another embodiment provides for compositions and methods to modulate calcium homeostasis in the ER. More specifically, increased SERCA concentration or activity in the hepatic ER improves calcium homeostasis in the ER, and suppresses glucose production and thus restores normoglycemia. SERCA may be modulated using, for example, liver-specific SERCA agonists, phospholamban inhibitors, vitamin D interventions, as well as other genetic and molecular approaches. Correcting SERCA function is also useful in suppressing hepatic VLDL production and dislipidemia, and thus atherosclerosis. Thus, an embodiment of the invention is a method for treating atherosclerosis or dislipidemia, or suppressing hepatic VLDL comprising modulating expression or activity of hepatic SERCA.

Yet another embodiment provides for the measurement of ASGAR and HP as diagnostic biomarkers for fatty liver disease and/or liver failure associated with ER stress and abnormal calcium metabolism. In particular, the synthesis of ASGAR and HP are dramatically reduced in the fatty liver as compared with normal liver.

### DESCRIPTION OF THE DRAWINGS

FIGS. 1A to 1E present the proteomic and lipidomic landscape of the lean and obese ER. FIG. 1A shows biological pathways associated with significantly regulated proteins in the obese ER proteome. Bar colors indicate the fold enrichment with significance values (negative log of p-values) superimposed. FIGS. 1B, 1C show transcript levels of genes involved in lipid metabolism in the lean and obese mouse liver. FIG. 1D shows alterations of liver ER lipidome. Heatmap display of all significant (p<0.05) alterations present between lean and obese ER lipidomes. The color corresponds to differences in the relative abundance (nmol %) of each fatty acid among individual lipid groups detected in the lean and obese liver ER. FIG. 1E shows the relative abundance of PC and PE in lean and obese liver ER samples. Values are mean±SEM n=6 or each roup). * denotes p<0.05, Student's t-test.

FIGS. 2a-2h demonstrate that elevated PC/PE ratio impairs SERCA activity and ER homeostasis. FIG. 2a reflects calcium transport activity of microsomes loaded with PC and PE in vitro. Transcript levels of Pemt (FIG. 2b) and corresponding microsomal calcium transport activities (FIG. 2c) of Hepa1-6 cells expressing control (Gfp) or mouse Pemt ORF. FIG. 2d shows calcium transport activity (top) and SERCA protein levels (bottom) of microsomes prepared from lean and obese mouse liver. Liver Serca2b transcript levels (FIG. 2e) and microsomal calcium transport activities (FIG. 2f, immunoblot (FIG. 2g) and quantitative RT-PCR (FIG. 2h) measurement of ER stress markers in the livers of lean mice expressing either LacZ (control) or Serca2b shRNAs. * in FIG. 2h denotes the phosphorylated IRE1a; and * in other panels denotes significant difference (p<0.05, n=4) by student's t-test. Values are mean±SEM.

FIGS. 3a-3l show that suppression of liver Pemt expression corrects ER PC/PE ratio, relieves ER stress, and improves systemic glucose homeostasis in obesity. FIG. 3a, PC/PE ratio, and FIG. 3b, calcium transport activity of liver ER from ob/ob mice expressing LacZ (control) or Pemt shRNAs. Immunoblot (FIG. 3c) and quantitative PCR (FIG. 3d) measurement of ER stress markers in the liver. Expression of hepatic lipogenesis and gluconeogenesis genes (FIG. 3e), triglyceride content (FIG. 3c, and Hematoxylin & Eosin staining (FIGS. 3g and 3h) of liver samples. Plasma glucose (FIG. 3i) and insulin (FIG. 3j) levels in control and Pemt shRNA-treated ob/ob mice after 6-hour food withdrawal. FIGS. 3k-3l, Plasma glucose levels of control and Pemt shRNA-treated ob/ob mice after intraperitoneal administration of either 1 g/kg of glucose (FIG. 3k) or 1 IU/kg of insulin (FIG. 3l). All data are mean±EM (n=4 for 3a-3e, n=6 for 3f-3l); * denotes p<0.05 (one-way ANOVA for data presented in 3k and 3l, and Student's t-test for others).

FIGS. 4a-4i demonstrate exogenous SERCA expression alleviates ER stress and improves systemic glucose homeostasis. Liver Serca2b transcript levels (4a) and microsomal calcium transport activities (4b) of control or Serca2b overexpressing obese mice. Plasma glucose (4c) Plasma insulin levels (4d), tissue weights (4e) of ob/ob mice as in panel a. Triglyceride content (4f, H&E staining (4g, 4h) and immunoblot analyses (4i) of ER stress markers (IRE1a and eIF2a phosphorylation, and CHOP) and secretory proteins (ASGR and HP) in the obese liver expressing Serca2b compared to controls. All values are mean±SEM (n=4 for 4a-4b, n=6 for 4c-4h); * denotes p<0.05 (Student's t-test).

FIGS. 5A-5D present data from ER fractionation and validation. FIG. 5A, is an illustration of ER fractionation procedure for proteomic and lipidomic analyses and polysome profiling. FIG. 5B shows validation of ER fractionation methodology by immunoblot analyses of subcellular markers. PDI: protein disulfide isomerase, CANX: Calnexin, IR: Insulin receptor, H2A: Histone 2A. FIG. 5C is a volcano plot of the fold changes of median spectral counts of proteins from obese and lean samples against the significance of differential expression (log-normalized p-Values). Proteins of interest are highlighted (red: p<0.05, fold of change (obese/lean) ˜1.5, average spectral counts ˜5; green: p<0.05, fold of change (lean/obese) ˜1.5, average spectral counts ˜5). FIG. 5D shows immunoblot of differentially regulated proteins identified from the proteomic study for protein lysates prepared from cytosolic and ER fractions of unfasted lean and obese liver. PMSA: Proteasome small subunit a, RPS6: Ribosomal small subunit 6, APOB: Apolipoprotein B, Mtp: Microsmal triglyceride transfer protein; HP: Hepatoglobin; ASGR: Asialoglycoprotein receptor; mEH: Microsomal epoxide hydrolase; MRC1: Mannose receptor, C type 1.

FIG. 6A-6B show expression of ER stress markers in the obese liver. FIG. 6a, Immunoblot detection of representative ER stress markers in total protein lysates prepared from the liver of lean and ob/ob mice sacrificed at 12 weeks of age after 6 hours of food withdrawal. FIG. 6b, Transcript levels of genes involved in ER-associated protein degradation (ERAD) in the liver of lean and ob/ob mice as determined by quantitative RT-PCR.

FIGS. 7A-7C demonstrate the distinct contributions of dietary fat and de novo lipogenesis to ER lipid composition. FIG. 7A is an illustration of the synthesis of nine classes of lipids detected in the ER lipidome. Dashed lines indicate multiple enzymetic steps. Genes studied herein are colored red. FIG. 7B is a heatmap display of all significant (p<0.05, Student's t-test) alterations present between diet and lean ER lipidomes. The color scheme reflects differences calculated based on the relative abundance (nmol %) of each fatty acid among individual lipid groups detected in the ER of lean liver and the diet. FIG. 7C shows a complete linkage analysis of all twelve ER lipidomes (six lean vs. six obese). The length of each branch correlates with the magnitude of lipidomic differences.

FIGS. 8A-8D show the effect of Pemt knockdown on liver ER lipidome and ER stress in ob/ob mice. FIG. 8A, Transcript levels of Pemt in the liver of ob/ob mice administered with adenoviral control (LacZ shRNA) or Pemt shRNA expressing viruses. FIG. 8B, Heatmap display of the fatty acid composition of ER isolated from the liver of ob/ob mice administered with control and Pemt shRNA. The color scheme denotes differences calculated from the relative abundance (nmol %) of each fatty acid among individual lipid groups detected in the ER of control and Pemt shRNA liver samples. FIG. 8C, Complete linkage analysis of ER lipidome for samples prepared from control and experimental groups. FIG. 8d, Quantification of immunoblot signals presented in FIG. 3d. Values are mean±SEM; n=4; * denotes p<0.05, Student's t-test.

FIGS. 9A-9E demonstrate amelioration of ER stress in the liver of high-fat diet (HFD) induced obese mouse by Pemt knockdown. FIGS. 9A-9B, Hematoxylin & Eosin staining of liver sections prepared from control (FIG. 9A) as well as Pemt shRNA-treated mice after 22 weeks of HFD (FIG. 9B). The white vesicles represent lipid droplets. FIG. 9C, Blood glucose levels of control and Pemt shRNA-treated HFD mice. FIGS. 9D-9E, Immunoblot and quantification of ER stress markers in the liver of control and experimental HFD mice. Values are mean±SEM, n=4; * denotes p<0.05, Student's t-test.

FIGS. 10A-10B show that SERCA2b overexpression improves systematic glucose homeostasis of ob/ob mice. Plasma glucose levels of control and SERCA2b overexpressing ob/ob mice after intraperitoneal administration of either 1 IU/kg of insulin (FIG. 10A) or 1 g/kg of glucose (FIG. 10B). All data are mean±SEM; * denotes p<0.05 (one-way ANOVA, n=6/group).

FIGS. 11A-11E show detergent-dependent solubilization of SERCA2b proteins from fatty liver samples and comparison of SERCA2b expression in lean with obese animals. FIG. 11a, Immunoblot of total protein lysates as well as ER fractions prepared from the liver of lean and obese mice following two different solubilization methods from the same samples. Liver tissue was first homogenized in lysis buffer containing 1% NP40 and clarified at 200 g for 10 minutes to pellet down cell debris. The whole cell lysate was either further solubilized by the addition of Laemmli buffer (2% SDS, top panel) or clarified by consecutive centrifugations at 16,000 g for 10 minutes and 60 minutes (middle panel) as described (see Park et al., 107 PNAS 19230 (2010)), supernatant collected, boiled in Laemmli buffer and loaded on to SDS-PAGE. For the examination of SERCA2b protein levels in the liver ER (bottom panel), ER pellet was resuspended in Laemmli buffer (2% SDS), sonicated for 3 minutes, boiled and clarified by centrifugation at 10,000 g for 10 minutes. FIGS. 11b-11c, Transcript levels of Serca2b in the liver tissues of genetically obese (12 weeks old, 11b) and diet-induced obese (22 weeks of HFD) mice as compared to age-matched lean controls. FIGS. 11d-11e, SERCA2b protein levels in the liver tissues of genetically obese as well as diet-induced obese mice at different ages. The total protein lysates were prepared with Laemmli buffer containing 2% SDS as described in the Examples.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The present embodiments address the discovery that there is a fundamental shift in hepatic endoplasmic reticulum (ER) function in obesity: from protein to lipid synthesis and metabolism. The presented invention demonstrates that modulating (i.e., correcting) hepatic calcium homeostasis and/or ER phospholipid synthesis suppresses hepatic glucose production, increases hepatic lipid oxidation, decreases hepatic VLDL production, and thus improves dislipidemia, and most importantly, normalizes systematic glucose levels and normoinsulinemia. The role of modulating hepatic lipid metabolism and/or calcium homeostasis in restoring systematic normoglycemia and normoinsulinemia, and the role of calcium homeostasis in suppressing hepatic VLDL production and thus dislipidemia (and atherosclerosis) provide novel approaches for treating many liver disease states associated with obesity.

The ER is the main site of protein and lipid synthesis, membrane biogenesis, xenobiotic detoxification and cellular calcium storage. Perturbation of ER homeostasis leads to stress and the activation of unfolded protein response (UPR). Ron & Walter, 8 Nat. Rev. Mol. Cell. Bio. 519 (2007). Chronic activation of ER stress has been shown to play an important role in the development of insulin resistance and diabetes in obesity. Hotamisligil, 140 Cell, 900 (2010). Mechanisms that lead to chronic ER stress in a metabolic context in general, and obesity in particular, remained a mystery until the present invention. Herein, comparative examination the proteomic and lipidomic landscape of hepatic ER purified from lean and obese mice reveal the mechanisms of chronic ER stress in obesity: Suppression of protein but stimulation of lipid synthesis in the obese ER occurs without significant alterations in chaperone content. Alterations in the ER fatty acid and lipid composition results in the inhibition of sarco/endoplasmic reticulum calcium ATPase (SERCA) activity and ER stress. Correcting the obesity-induced alteration of ER phospholipid composition or hepatic SERCA overexpression in vivo both reduced chronic ER stress and improved glucose homeostasis. Hence, the present inventors have discovered that abnormal lipid and calcium metabolism are important contributors to hepatic ER stress in obesity.

It has been generally accepted that a surplus of nutrients and energy stimulates synthetic pathways and may lead to client overloading in the ER. It has not been demonstrated, however, whether increased de novo protein synthesis and client loading into the ER and/or a diminished productivity of ER in protein degradation or folding leads to ER stress in obesity. Intriguingly, dephosphorylation of eukaryotic translation initiation factor 2a (eIF2a) in the liver of high-fat-diet fed mice reduced ER stress response (Oyadomari et al., 7 Cell Metab. 520 (2008)), suggesting that additional mechanisms other than translational up-regulation may also contribute to ER dysfunction in obesity.

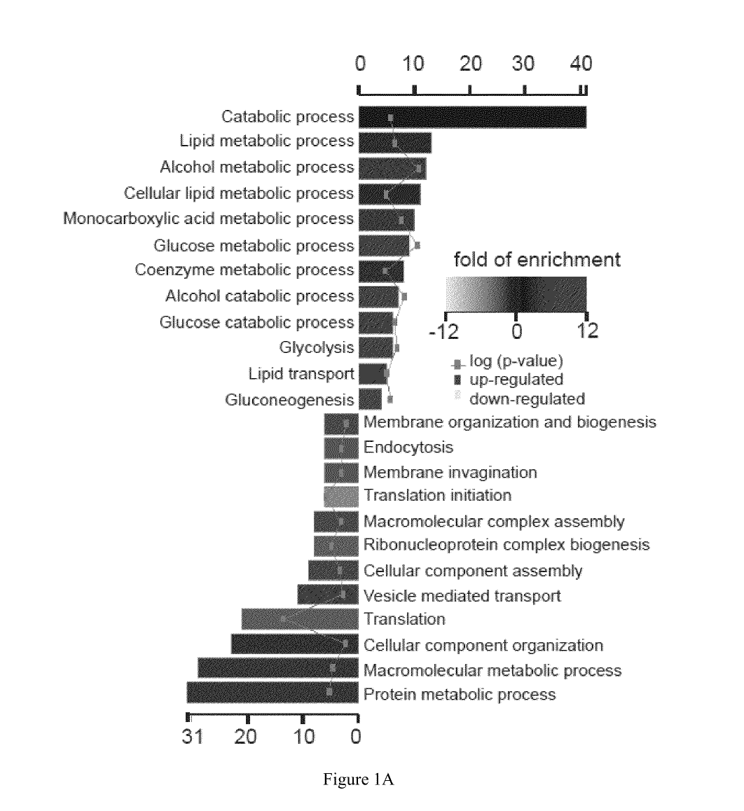

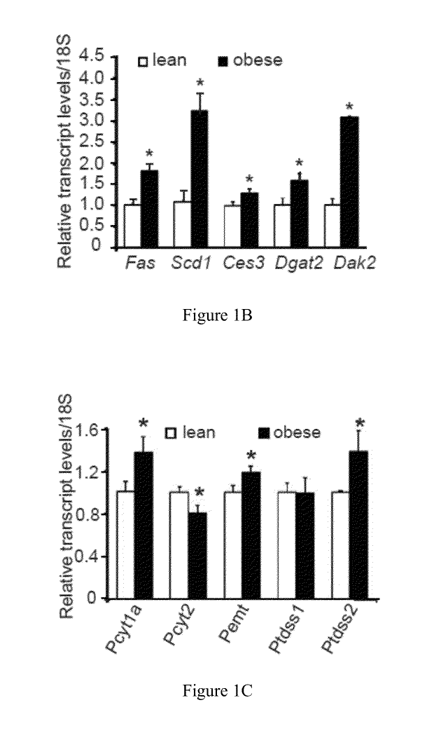

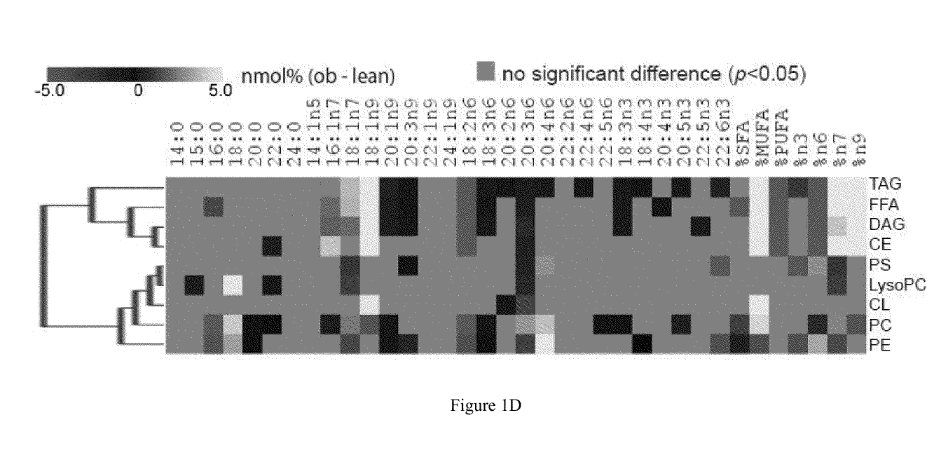

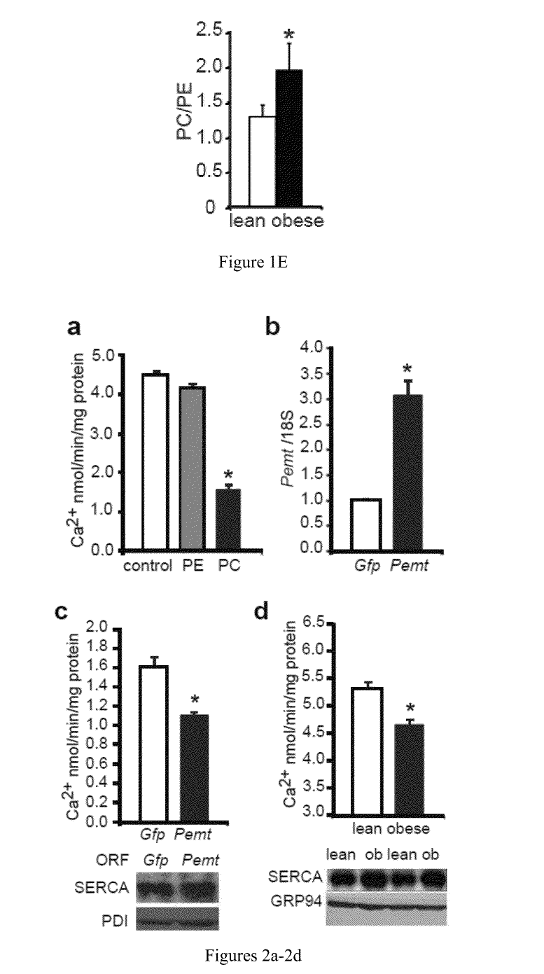

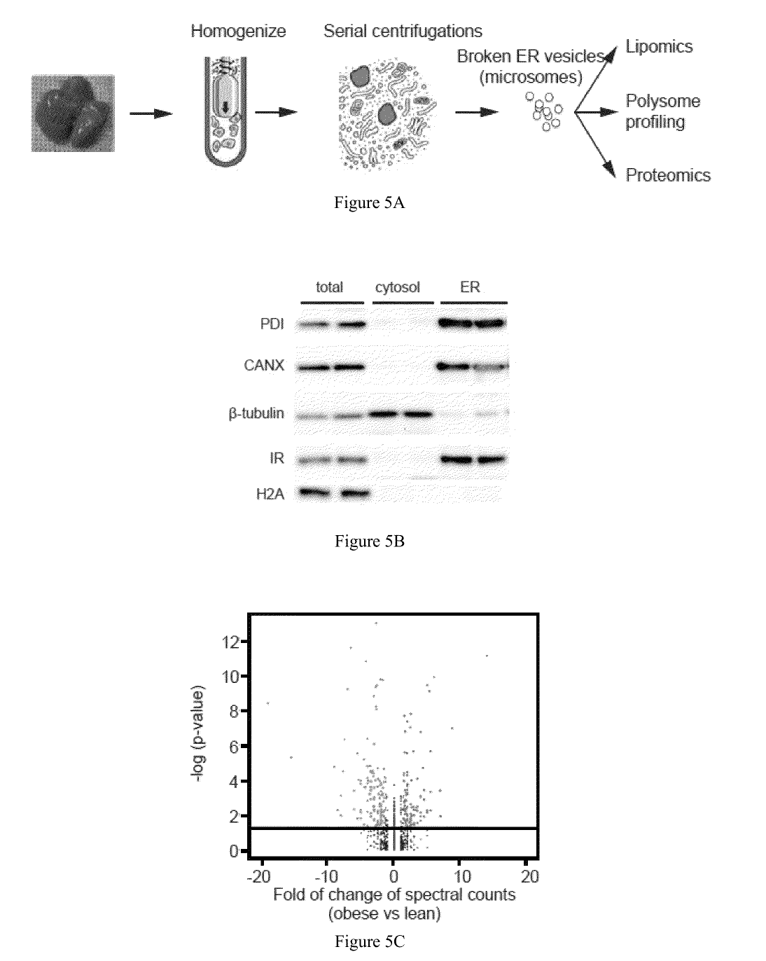

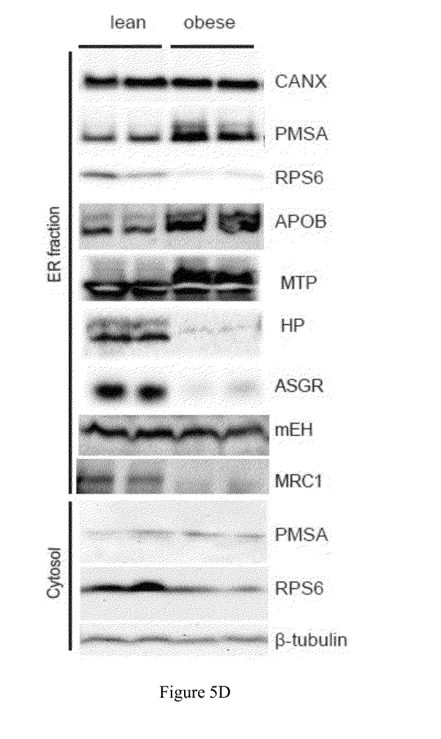

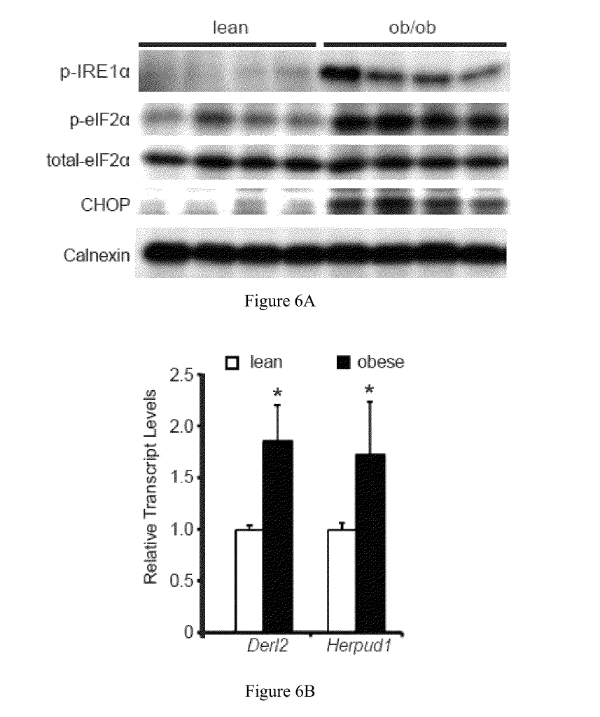

To address these mechanistic questions, ER was fractionated from lean and obese liver tissues (FIGS. 5A-5B) and then extracted ER proteins for comparative proteomic analysis to examine the status of this organelle in obesity. A total of 2,021 unique proteins were identified. Among them, 120 proteins were differentially regulated in obese hepatic ER samples (FIG. 5C, Tables 1a and 1b). The differential regulation was validated, when possible, by immunoblot analyses, and the fidelity of the system verified (FIG. 5D). Gene Ontology analysis identified the enrichment of metabolic enzymes, especially ones involved in lipid metabolism, in the obese ER proteome, while protein synthesis and transport functions were over-represented among down-regulated ER proteins (FIG. 1A). Consistently, ER associated protein synthesis was down-regulated in the obese liver as demonstrated by polysome profiling, whereas the expression of genes involved in de novo lipogenesis (Fas, Scd1, Ces3, Dgat2 and Dak2) and phospholipid synthesis (Pcyt1a and Pemt) were broadly up-regulated (FIGS. 1B, 1C). Many components of protein degradation pathways were also upregulated, with no broad change in the quantity of ER chaperones (FIGS. 6A-6B, Table 1 a). Taken together, these data revealed a fundamental shift in hepatic ER function in obesity from protein to lipid synthesis and metabolism.

The presence of chronic ER stress in obese liver (FIGS. 6A-6B) despite reduction in ER-associated protein synthesis led to the hypothesis that ER stress in obesity may not be invoked simply by protein overloading, but is also driven by compromised folding capacity influenced by lipid metabolism. Erbay et al., 15 Nat. Med. 1383 (2009). For example, the ability of palmitate and cholesterol to induce ER stress in cultured cells correlates with their incorporation into the ER. Li et al., 270 J. Biol. Chem. 37030 (2004); Borradaile et al., 47 J. Lipid Res. 2726 (2006).

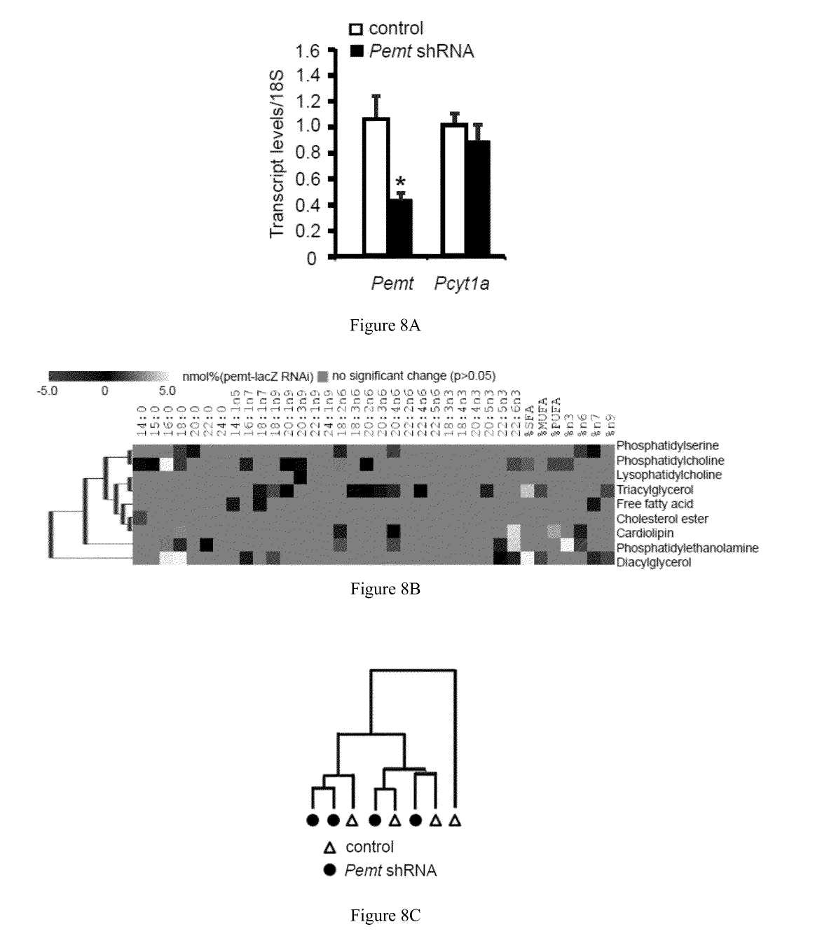

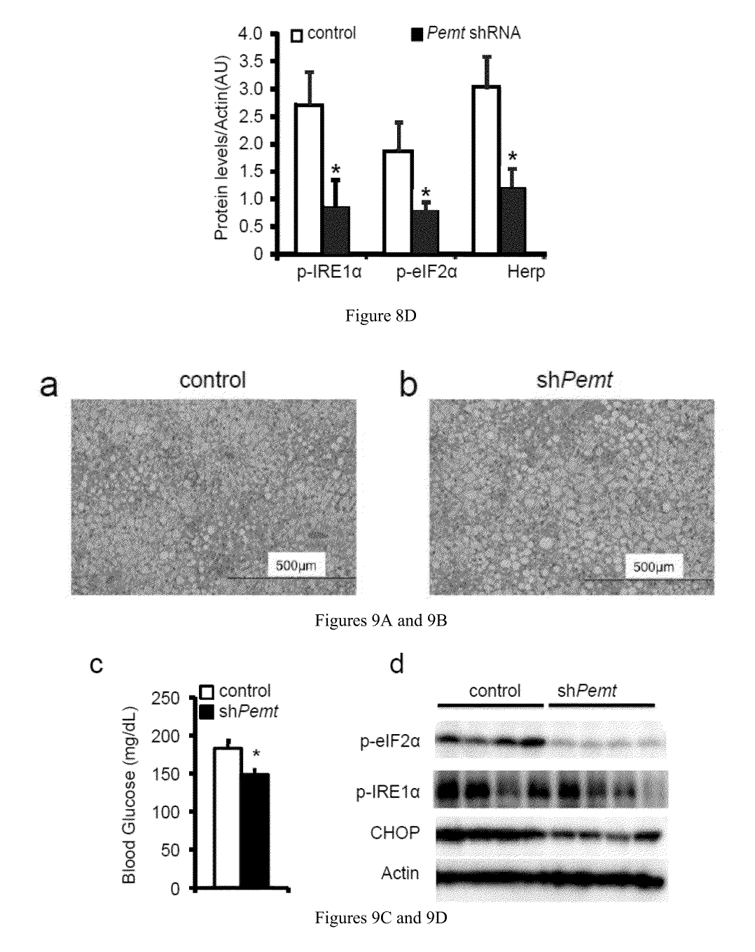

Therefore, a quantitative determination of all major lipid species and their fatty acid composition in ER samples isolated from lean and obese liver along with the diet consumed by these animals was undertaken. (FIG. 8A-8D, Table 2). This revealed that the fatty acid composition of ER lipids in the lean mouse liver was distinct from corresponding dietary lipids, suggesting the contribution of a basal level de novo lipogenesis to the biogenesis of ER membranes in vivo (FIGS. 6a, 6b; Table 2). Almost all ER derived lipids were composed of significantly higher levels of saturated fatty acids (SFA) whereas their polyunsaturated fatty acid (PUFA) content was much lower than those of corresponding dietary lipids, suggesting that de novo synthesized SFAs are preferred over diet-derived PUFAs as the substrate for the synthesis of hepatic ER lipids. Additionally, the liver ER samples of lean and obese mice also had profoundly different composition of fatty acids and lipids as illustrated by the clear separation of lean and obese ER lipidome in cluster analysis (FIG. 1D). The obese ER was significantly enriched with monounsaturated fatty acids (MUFA, FIG. 1E), a bona fide product of de novo lipogenesis in liver.

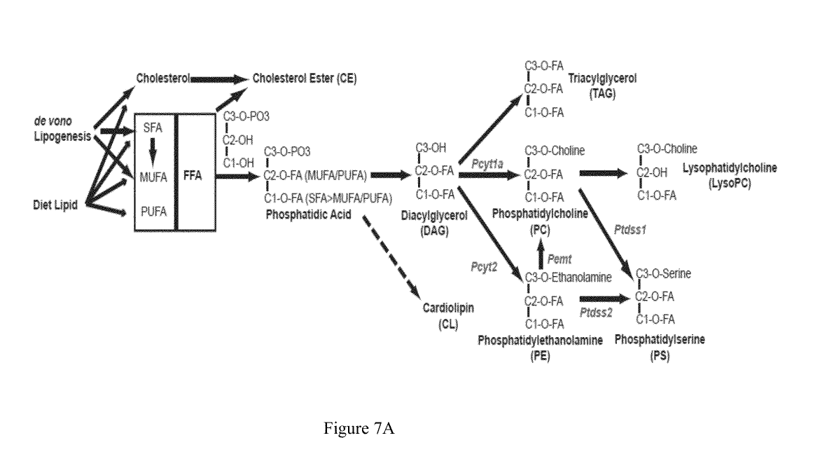

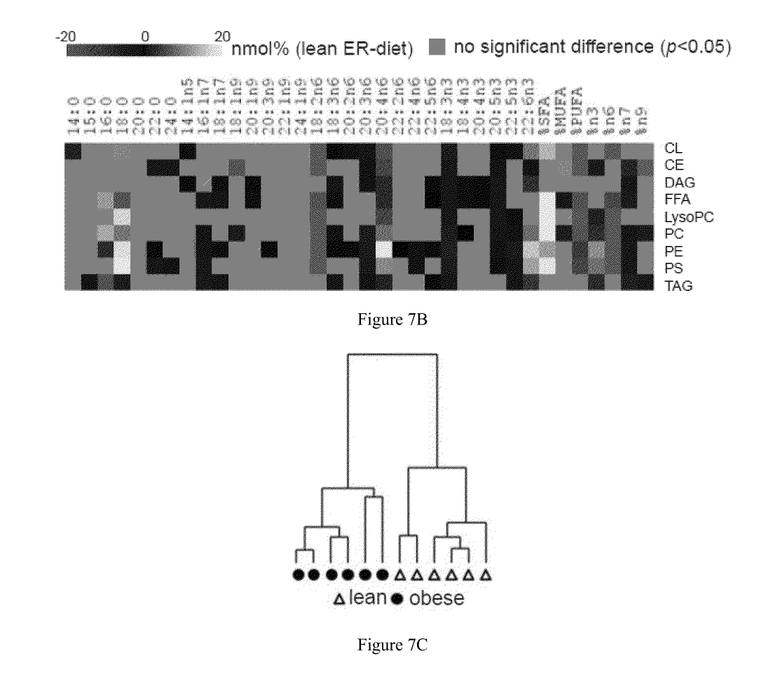

Importantly, the obese ER samples contained a higher level of phosphatidylcholine (PC) as compared to phosphatidylethanolamine (PE) (PC/PE=1.97 vs. 1.3, p<0.05, Table 2), two of the most abundant phospholipids on the ER membrane. The rise of PC/PE ratio is likely caused by the up-regulation of two key genes involved in PC synthesis and PE to PC conversion: choline-phosphate cytidylyltransferase A (Pcyt1a) and phosphatidylethanolamine N-methyltransferase (Pemt) (FIG. 1C, FIG. 7a), and it is consistent with the essential role of PC for lipid packaging in the form of lipid-droplets or lipoproteins, both of which are increased in obesity. In contrast, the PC/PE ratio in the lean hepatic ER was essentially identical as it is in the diet (Table 2), indicating that the increase of PC/PE ratio in obesity is not due to food consumption, but the result of increased lipid synthesis in the obese liver.

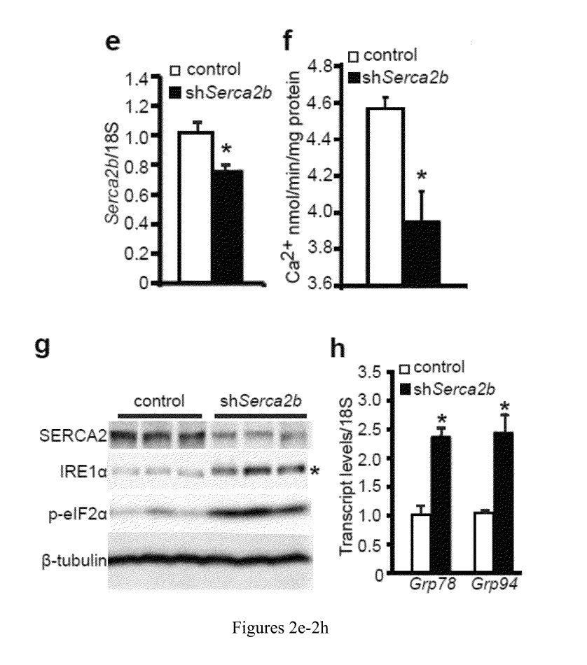

The desaturation of SFA to MUFA in the obese liver likely has a protective role in reducing lipotoxicity, whereas the decrease of PUFA content in the ER may limit its reducing capacity and contribute to ER stress. Kim, 479 Neurosci. Lett. 292 (2010). The role of PC/PE ratio in regulating hepatic ER homeostasis has not been studied before. Previous biochemical studies have shown that increasing PC content in the membrane inhibits the calcium transport activity of SERCA 5,8. Li et al., 2004; Cheng et al., 261 J. Bio. Chem. 5081 (1986). Consistently, it was found herein that the addition of PC to liver-derived microsomes in vitro substantially inhibited SERCA activity (FIGS. 2A, 2B). More importantly, overexpression of the PE to PC conversion enzyme, Pemt, in Hepa1-6 cells significantly inhibited microsomal SERCA activity, suggesting changes in the PC/PE balance in a cellular setting can significantly perturb SERCA function (FIGS. 2C, 2D). Because calcium plays an important role in mediating chaperone function and protein folding in the ER, and given that SERCA is principally responsible in maintaining calcium homeostasis in this organelle, it was postulated that the increased PC/PE ratio in the ER of obese liver might impair ER calcium retention and homeostasis in vivo, thereby contributing to protein misfolding and ER stress. Indeed, as shown herein, microsomes prepared from obese mice livers had significantly lower calcium transport activity than those isolated from lean animals (4.60.2 vs. 5.30.3, p=0.046, FIG. 2e), despite the fact that SERCA protein level was modestly higher in the former: consistent with an inhibitory role of PC/PE ratio on SERCA function.

Although SERCA dysfunctions have been reported in the muscle of diabetic patients, its role in hepatic ER stress, as shown herein, is novel. Modest defects in SERCA activity have been implicated in the pathology of Darier's disease (Miyauchi et al., 281 J. Biol. Chem. 22882 (2006)). It was found herein that a reduction in SERCA expression in vivo (FIG. 2f) and a concurrent reduction in its calcium transport activity (FIG. 2g) potently activated hepatic ER stress in lean mice as evident by IRE1a and eIF2a phosphorylation and changes in the expression of Grp78 and Grp94 (FIG. 2h). Therefore, there appears to be little redundancy in the function of SERCA beyond physiological fluctuations to maintain ER homeostasis, and the reduction in calcium transport activity is a potential mechanism of hepatic ER stress in obesity.

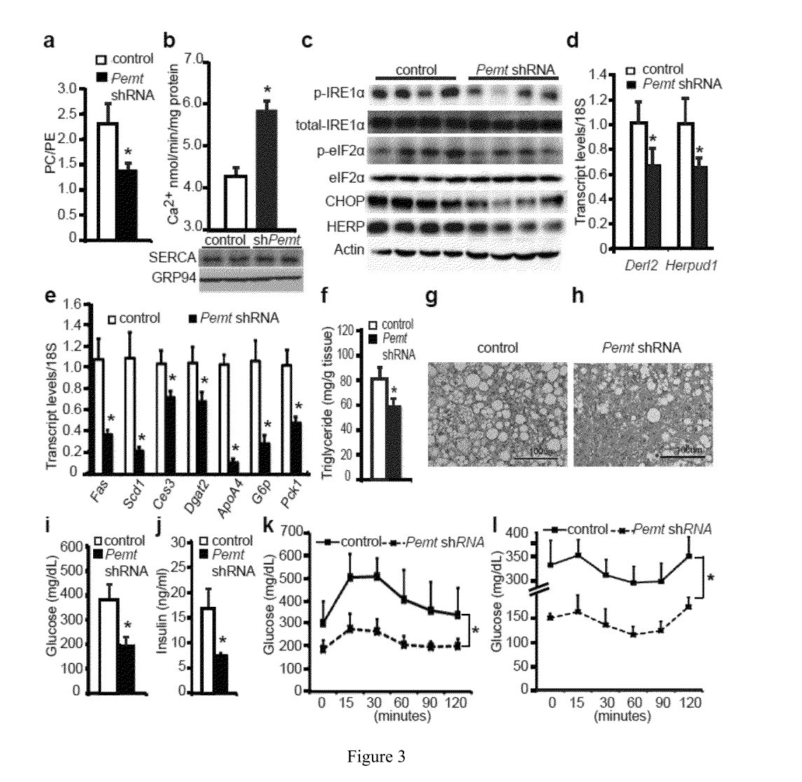

Different but complementary approaches to correct aberrant lipid metabolism caused SERCA dysfunction and the effects on ER homeostasis in the obese liver were examined. If the alteration in PC/PE ratio seen in obese liver is a significant contributor to ER stress, correction of this ratio to lean levels by reducing Pemt expression should improve calcium transport defects and produce beneficial effects on hepatic ER stress and metabolism. An adenovirally-expressed shRNA system achieved ˜50-70% suppression of the Pemt transcript in obese liver (FIG. 3A). As postulated, suppression of Pemt led to a decrease of PC content from ˜39% to ˜33%, which was compensated by an ˜7% increase of PE content from ˜17% to 24% (Table 3). As a result, the PC/PE ratio is reduced to 1.3 (equivalent to lean ratio), as compared to 2.0 detected in the ER of the obese liver (FIG. 3A). The reduction of PC/PE ratio was accompanied by a significant improvement in the calcium transport activity of the ER prepared from the Pemt-knockdown obese mice (FIG. 3B). As the improvement of calcium transport function occurred with few and minor changes in the overall fatty acid composition of ER (FIGS. 8A, 8B; Table 3), these results confirmed the rise in PC/PE ratio as an inhibitory factor of SERCA activity in obesity.

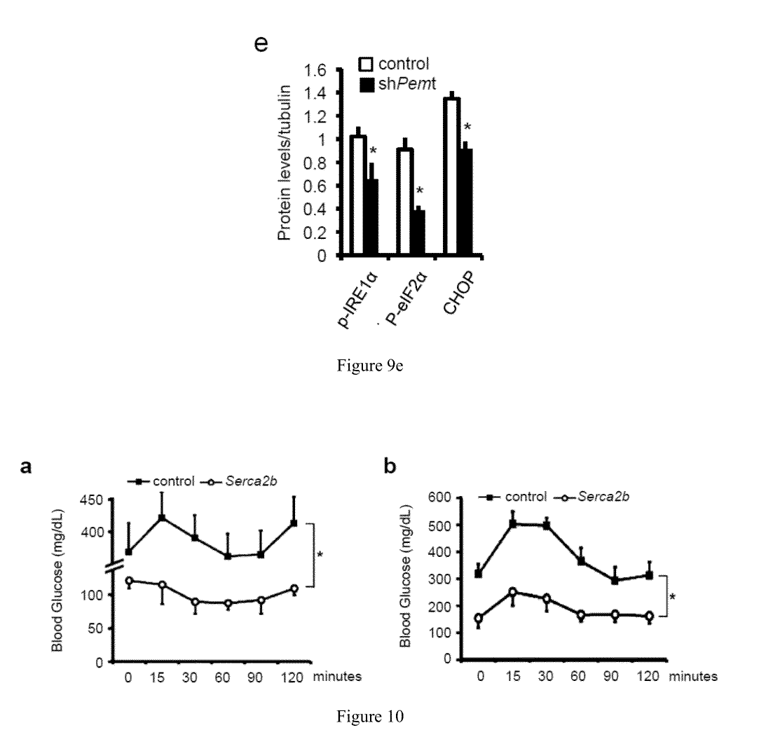

More importantly, hepatic ER stress indicators including the phosphorylation of IRE1a and eIF2a, as well as the expression of C/EBP homologous protein (CHOP), homocysteine-inducible, endoplasmic reticulum stress-inducible protein (HERP) and Der1-like domain family member 2 (DERL2), were all reduced upon suppression of Pemt in obese mice (FIGS. 3C, 3D; FIG. 8C). Relief of chronic ER stress in the ob/ob mice has been associated with improvement of hepatic steatosis and glucose homeostasis, and Pemt knockout mice have been shown to be protected from diet-induced dislipidemia. Ozcan et al., 313 Sci. 1137 (2006); Kammoun et al., 119 J. Clin. Investig. 1201 (2009). It was found herein that genes involved in hepatic lipogenesis (Fas, Scd1, Ces3, Dgat2) and lipoprotein synthesis (ApoA4) were consistently and significantly down-regulated in the obese liver following suppression of Pemt (FIG. 3E). As a result, these mice exhibited a significant reduction in hepatic steatosis and liver triglyceride content (FIGS. 3F-3H). Genes involved in glucose production (G6p, Pck1) in the liver were significantly down-regulated (FIG. 3E), and there were also significant reductions in both hyperglycemia and hyperinsulinemia in obese mice following the suppression of hepatic Pemt expression (FIGS. 3I, 3J). Glucose and insulin tolerance tests revealed significantly enhanced glucose disposal following Pemt suppression (FIG. 3K, 3L). A similar phenotype is also observed upon suppression of hepatic Pemt in the high-fat diet induced obesity with reduced ER stress and improved glucose homeostasis (FIGS. 9A-9D). These data are consistent with the phenotype seen in Pemt-deficient mice, which exhibit protection against diet-induced insulin resistance and atherosclerosis. Jacobs et al., 285 J. Biol. Chem. 22403 (2010). Therefore, correcting the PC/PE ratio of ER can significantly improve calcium transport defects, reduce ER stress and improve metabolism, supporting the hypothesis that changes in lipid metabolism contribute to SERCA dysfunction, ER stress and hyperglycemia in both genetic- and diet-induced models of obesity.

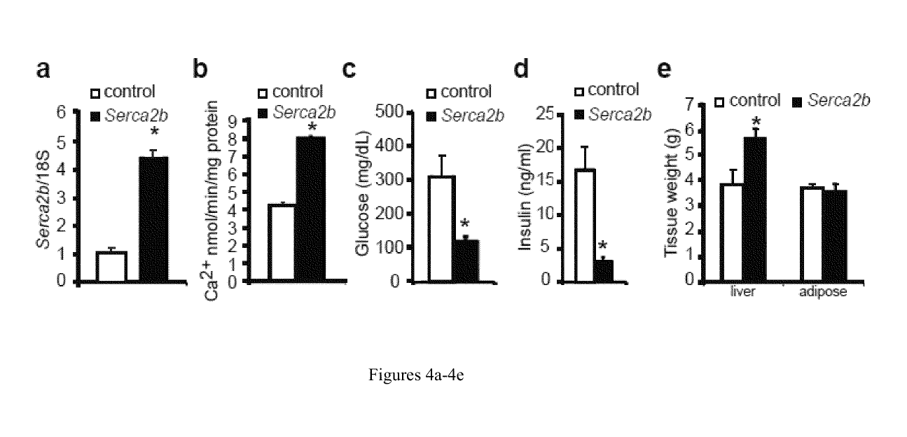

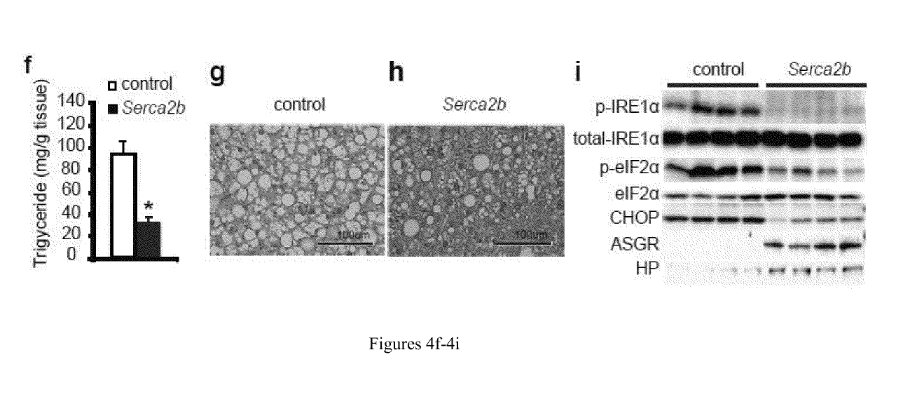

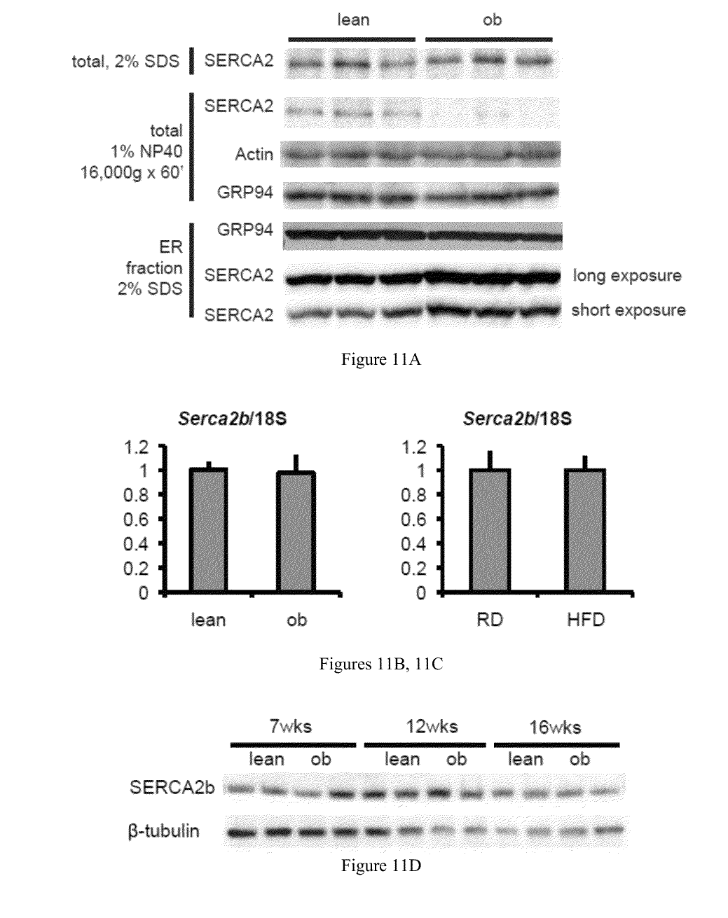

Additionally, over-expression of hepatic Serca in vivo, to overcome the partial inhibition of SERCA activity by PC (FIG. 4a) showed that exogenous SERCA expression in the liver of the ob/ob mice improved the calcium import activity of the ER (FIG. 4b), restored euglycemia and normoinsulinemia within a few days, and markedly improved glucose tolerance (FIGS. 4C, 4D; FIGS. 10A-10B). Upon Serca expression, liver showed an increase in size but a marked reduction of lipid infiltration (FIGS. 4E-4H) and suppression of IRE1a and eIF2a phosphorylation, along with significant reduction in CHOP levels (FIG. 4I). In these liver samples, there was also a marked increase in two secretory proteins that were otherwise diminished in obesity: asialoglycoprotein receptor (ASGR) and haptoglobin (HP) (FIG. 4I). As the folding and maturation of ASGR is sensitive to perturbations of calcium homeostasis in the ER (Lodish & Kong, 265 J. Biol. Chem. 10893 (1990)), the results herein support that exogenously increased SERCA expression restored calcium homeostasis and relieved at least some aspects of chronic ER stress in the obese liver. Taken together, these data reinforced the hypothesis that lipid-driven alterations and the ER calcium homeostasis are important contributors to hepatic ER stress in obesity.

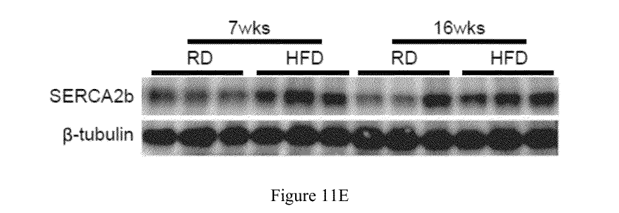

The chronic activation of ER stress markers has been observed in a variety of experimental obese models as well as in obese humans. Gregor et al., 58 Diabetes 693 (2009). Furthermore, treatment of obese mice and humans with chemical chaperones result in increased insulin sensitivity. Ozcan et al., 2006; Kars et al., 59 Diabetes 1899 (2010). The present systematic, compositional and functional characterization of hepatic ER landscape from lean and obese mice revealed a diametrically opposite regulation of ER functions regarding protein and lipid metabolism and revealed mechanisms giving rise to ER stress. In particular, elevation of the PC/PE ratio in the ER, driven by the up-regulation of de novo lipogenesis in obesity, was linked to SERCA dysfunction and chronic ER stress in vivo. A recent study reported down-regulation of SERCA protein level in obese liver (Kars et al., 2010), which was not evident in our analysis and appeared to have resulted from the choice of methodology in ER protein preparations (FIGS. 11A-11E). Nevertheless, other mechanisms such as oxidative and inflammatory changes associated with obesity can also perturb ER homeostasis by impacting ER calcium fluxes. See, e.g., Park et al., 107 PNAS 19320 (2010); Li et al., 49 Diabetologia 1434 (2006); Cardozo et al., 54 Diabetes 452 (2005).

The identification of a lipid-driven calcium transport dysfunction and ER stress provides a fundamental framework to understand the pathogenesis of hepatic lipid metabolism and chronic ER stress in obesity. Excessive food intake inevitably stimulates lipogenesis for energy storage, and PC is the preferred phospholipid coat of lipid droplets and lipoproteins. Li et al., 186 J. Cell. Bio. 783 (2009). Therefore, there is a biological need for the synthesis of more PC for packaging and storing the products of hepatic lipogenesis. Also, de novo fatty acid synthesis in the obese liver produces ample amounts of MUFA, which is effectively incorporated into PC but not PE, which further distorts the PC/PE ratio and impairs ER function. The resulting ER stress facilitates the secretion of excessive lipids from liver without ameliorating hyperinsulinemia-induced lipogenesis (Schiller et al., 42 J. Lipid Res. 1501 (2001)), and thus hepatosteatosis and ER stress ensue. As a result relieving ER stress in obesity may ultimately depend on breaking this “lipogenesis-ER stress-lipogenesis” vicious cycle and restoring the ER folding capacity. Therefore, genetic, chemical or dietary interventions that modulate hepatic phospholipid synthesis and/or ER calcium homeostasis function represent a new set of therapeutic opportunities for common chronic diseases associated with ER stress such as obesity, insulin resistance, and type 2 diabetes.

The interventions that modulate hepatic phospholipid synthesis and/or ER calcium homeostasis function may be used as treatment of hepatic ER stress-associated disease states including type 2 diabetes, dislipidemia, fatty liver disease, inflammation, and/or atherosclerosis. Such treatment may improve a diagnosed condition or make it more manageable, or improve disease symptoms, or correct physiological imbalances associated with hepatic ER stress. Treatment can also include delaying or preventing the onset of hepatic ER stress-associated disease, or preventing recurrence or relapse of hepatic ER stress-associated disease. For example, a treatment of hepatic ER stress improves glucose homeostasis.

In specific embodiments, the PC/PE ratio of the hepatic ER is modulated by inhibiting (or down-regulating) expression or activity of phosphatidylethanolamine N-methyltransferase (PEMT), encoded by Pemt. The modulating includes genetic, chemical or dietary intervention. An approach to inhibiting expression or activity of PEMT includes (optionally) identifying a cell, cell population or tissue in which modulation (reduction) of the activity or level of PEMT is desired; and contacting said cell, cell population or tissue with an amount of PEMT modulator(s), e.g., PEMT antagonist(s), sufficient to modulate the activity or level of PEMT in the cell, cell population, or tissue. The contacting step may be carried out ex vivo, in vitro, or in vivo. For example, the contacting step may be performed using human cells, or performed in a subject such as a human patient. The PEMT inhibitor may be, for example, an anti-PEMT antibody, a portion of S-adenosyl-L-methionine or phosphatidylethanolamine that acts as a decoy for PEMT, or a small molecule inhibitor of PEMT. The antibody antagonist may be a monoclonal or single specificity antibody, may be human, humanized, chimeric, or in vitro generated antibody. The term antibodies also includes any portion of an antibody that binds to a PEMT epitope. An example chemical that inhibits PEMT is rosiglitazone, available as AVANDIA® (rosiglitazone maleate), AVANANAMET® (rosiglitazone maleate/metformin HCl) and AVANDARYL® (rosiglitazone maleate and glimepiride) from GlaxoSmithKline. Additional PEMT inhibitors include, for example, 3-deazaadenosine (DZA), bezafibrate and clofibric acid.

Alternatively, or in combination with PEMT inhibitors, expression of Pemt may be inhibited by RNA interference with, e.g., dsRNA, ssRNA, siRNA, shRNA, miRNA, and the like. In a particular embodiment, the RNA interference mediator is a shRNA, or a mixture of shRNAs. An example shRNA effective for inhibiting Pemt is presented in Table. 5.

Similarly, the PC/PE ratio of the hepatic ER can be modulated by inhibiting expression or activity of phosphate cytidylyltransferase 1, choline, alpha (also called choline-phosphate cytidylyltransferase A), encoded by Pcyt1a. The modulating includes genetic, chemical or dietary intervention. The nucleotide sequence of Pcyt1a is available, for example, at the National Center for Biotechnology Information (NCBI) website, ID: 5130 (Homo sapiens), as is Pemt, ID: 10400 (H. sapiens).

Additionally, because modulation of PEMT to down-regulate its expression or function was shown herein to down-regulate the expression of several other genes, additional or alterative modulators of these genes may be useful in the present invention to alleviate hepatic ER stress. Thus, this modulating comprises down-regulating hepatic expression of at least one of a de novo lipogenesis gene such as Fas, Scd1, Ces3, Dgat2 and Dak2; a lipoprotein synthesis gene ApoA4; or a gene involved in glucose production such asG6 and Pck1.

In other specific embodiments, the calcium homeostasis of hepatic ER is modulated by activating (or up-regulating) expression or activity of sarco(endo)plasmic reticulum Ca2+-ATPase (SERCA). The modulating includes genetic, chemical or dietary intervention. An approach to increasing expression or activity of SERCA includes (optionally) identifying a cell, cell population or tissue in which modulation (increase) of the activity or level of SERCA is desired; and contacting said cell, cell population or tissue with an amount of SERCA modulator(s), e.g., SERCA agonist(s), sufficient to modulate the activity or level of SERCA in the cell, cell population, or tissue. The contacting step may be carried out ex vivo, in vitro, or in vivo. For example, the contacting step may be performed using human cells, or performed in a subject such as a human patient.

Example chemical modulators that increase SERCA activity include nitroxides such as 4-Hydroxy-2,2,6,6-tetramethylpiperidine-N-oxyl (tempol), ursodeoxycholic acid, and tauroursodeoxycholic acid. Additional SERCA enhancers include, for example, istaroxime, NOS, TUDCA and regucalcin. Alternatively or in concert, SERCA concentration and activity can be increased by genetic means, (i.e., via gene therapy). Example genes encoding SERCA are available at NCBI, ID: 488, ID: 487, ID: 489 (each H. sapiens). Example primers for open reading frame (ORF) cloning are presented in Table 5. The viral vector delivery described herein can be modified for use in humans by techniques known in the art.

Gene therapy approaches that can be used to increase SERCA expression include lentivirus, herpesvirus, and nonviral vectors. See, e.g., Lam & Dean, Progress & prospects: nuclear import of nonviral vectors, 17 Gene Ther. 439 (2010); Macnab & Whitehouse, Progress & prospects: human artificial chromosomes, 16 Gene Ther. 1180 (2009); Epstein, Progress & prospects: Biological properties & technological advances of herpes simplex virus type 1-based amplicon vectors, 16 Gene Ther. 709 (2009); Brunetti-Pierri & Ng, Progress & prospects: gene therapy for genetic diseases with helper-dependent adenoviral vectors, 15 Gene Ther. 553 (2008); Sinn et al., Gene Therapy Progress & Prospects: Development of improved lentiviral & retroviral vectors—design, biosafety, & production, 12 Gene Ther. 1089 (2005); Flotte, Gene Therapy Progress & Prospects: Recombinant adeno-associated virus (rAAV) vectors, 11 Gene Ther. 805 (2004).

Additionally or alternatively, SERCA activity can be increased by inhibiting those mechanisms (e.g., lipids, proteins, or pathways) that remove SERCA from the hepatic ER. For example, phospholamban inhibitors can be used to maintain SERCA levels in the hepatic ER.

Vitamin and mineral supplements along with nutritional support may be useful in concert with any of the treatments discussed herein, including, for example, vitamin D interventions.

Additionally, the treatment or condition of the hepatic ER can be monitored by measuring expression of hepatic asialoglycoprotein receptor (ASGR) and/or haptoglobin (HP). Monitoring can be achieved using any approach known in the art, including PCR and immunoassay. Phospholamban inhibitors can be used as SERCA activators.

### EXAMPLES

### Example 1

### ER Fractionation from Obese and Lean Mice

Male leptin-deficient (ob/ob) and wild-type littermates in the C57BL/6J background were bred in-house and used for all biochemical experiments. Leptin deficient mice used for adenovirus-mediated expression experiments were purchased from the Jackson Laboratory (strain B6. V-Lepob/J, stock number 000632). All mice were maintained on a 12-hour-light/12-hour-dark cycle in a pathogen-free barrier facility with free access to water and regular chow diet containing 2200 ppm of choline (PicoLab® Mouse Diet 20).

ER fractionation protocols were adapted from Cox and Emili (1 Nat. Protoc. 1872 (2006)). Briefly, male mice at three months of age (unless otherwise noted) with or without overnight fasting were anesthetized by tribromoethanol and perfused with 20 ml 0.25 M sucrose solution before tissue harvesting. Fresh liver tissue (1.0 g for lean and 1.2 g for obese mice produced an equal amount of ER) was immediately transferred to 10 ml ice cold STM buffer (0.25 M sucrose, 50 mM Tris pH 7.4, 5 mM MgCl₂), chopped into small pieces and homogenized by 6 strokes in a motor-driven, loose-fit, teflon-glass homogenizer at speed setting of 3.5 (Wheaton, N.J.). The whole lysates were first cleared by centrifugation at 3000 g for 10 min followed by a series of centrifugations to obtain the final ER pellet. The pellet was washed with 11 ml of ice-cold 0.25M sucrose solution and was subjected to centrifugation to obtain the final ER preparation which was either snap frozen in liquid nitrogen or used directly for biochemical and other analysis.

### Example 2

### Sample Prefractionation by 1D-PAGE

Aliquots of 20 μl (˜100 pg) of the ER protein extract was boiled for 5 min in an equal volume of 2× Laemmli buffer and separated on a 12% SDS-poly-acrylamide gel (15 cm×15 cm×1.0 mm). The gel was minimally stained with Coomassie Brilliant Blue and briefly washed in 25% methanol, 7.5% acetic acid and sliced horizontally into 12 bands with roughly similar protein content as estimated from the optical density. See Schmidt et al., 3 Mol. Sys. Bio. 79 (2007). The gel was then cut vertically to separate the protein content of individual lanes. The gel slices were minced with a sterile clean razor blade, transferred into 96-well plates, washed three times with 200 μl of 25 mM ammonium bicarbonate 50% acetonitrile, followed by dehydration with 100 μl HPLC-grade acetonitrile. After removal of acetonitrile, the gel slices were dried completely in a vacuum concentrator (Speed Vac, Thermo, MA) and rehydrated in 200 μl of 50 mM ammonium bicarbonate containing 1 μg/ml trypsin, followed by incubation for 24 hr at 37° C. Protein digests were collected and the gel pieces were further extracted and washed (a) with 200 μl of aqueous 20 mM ammonium bicarbonate pH 8.6; (b) twice with 200 μl of 2% formic acid 50% HPLC-grade acetonitrile; followed by (c) dehydration in 150 μl of 2% formic acid 10% 2-propanol 85% acetonitrile. The combined peptide solutions were filtered using hydrophilic multi-well PTFE filter plates (Millipore, MA) according to the manufacturer's protocol and concentrated to a volume of ˜5 μl in a SpeedVac, and resuspended in 60 μl aqueous solvent containing 2% formic acid, 2% acetonitrile. Samples were analyzed by 1D nano-LC ESI tandem mass spectrometry as described herein.

### Example 3

### Protein Identification by 1 D Nano-LC Tandem Mass Spectrometry

LC MS/MS Instrumentation:

A CTC Autosampler (LEAP Technologies, NC) was equipped with two 10-port Valco valves and a 20 μl injection loop. A 2D LC system (Eksigent, CA) was used to deliver the flow rate of 3 μl/min during sample loading and 250 μl/min during nanoflow rate LC separation. Self-packed columns used: a C18 solid phase extraction “trapping” column (250 μm i.d.×10 mm) and a nano-LC capillary column (100 μm i.d.×15 cm, 8 μm i.d. pulled tip (NewObjective) both packed with the Magic C18AQ, 3 μm, 200 Å (Michrom Bioresources) stationary phase. A protein digest (10 μl) was injected onto the trapping column connected on-line with the nano-LC column through the 10-port Valco valve. The sample was cleaned up and concentrated using the trapping column, eluted onto and separated on the nano-LC column with a one-hour linear gradient of acetonitrile in 0.1% formic acid. The LC MS/MS solvents were Solvent A: 2% acetonitrile in aqueous 0.1% formic acid; and Solvent B: 5% isopropanol 85% acetonitrile in aqueous 0.1% formic acid. The 85-min-long LC gradient program included the following elution conditions: 2% B for 1 min; 2-35% B in 60 min; 35-90% B in 10 min; 90% B for 2 min; and 90-2% B in 2 min. The eluent was introduced into LTQ Orbitrap (ThermoElectron, CA) mass spectrometer equipped with a nanoelectrospray source (New Objective, MA) by nanoelectrospray. The source voltage was set to 2.2 kV and the temperature of the heated capillary was set to 180° C. For each scan cycle on full MS scan was acquired in the Orbitrap mass analyzer at 60,000 mass resolution, 6×10⁵ AGC target and 1200 ms maximum ion accumulation time was followed by 7 MS/MS scans acquired for the 7-most intense ions for each of the following m/z ranges 350-700, 695-1200, and 1195-1700 amu. The LTQ mass analyzer was set for 30,000 AGC target and 100 ms maximum accumulation time, 2.2 Da isolation width, and 30 ms activation at 35% normalized collision energy. Dynamic exclusion was enabled for 45 sec for each of the 200 ions that had been already selected for fragmentation to exclude them from repeated fragmentation. Each digest was analyzed twice.

MS Data Processing:

The MS data.raw files acquired by the LTQ Orbitrap mass spectrometer were copied to the Sorcerer IDAII search engine (Sage-N Research, Thermo Electron, CA) and submitted for database searches using the SEQUEST-Sorcerer algorithm. The search was performed against a concatenated FASTA protein database containing the forward and reversed human (25H. Sapiens) UniProt KB database downloaded from EMBL-EBI on Oct. 23, 2008 as well as an in-house compiled database with common contaminants. Methionine, histidine, and tryptophane oxidation (+15.994915 atomic mass units, amu) and cysteine alkylation (+57.021464 amu with iodoacetamide derivative) were set as differential modifications. No static modifications or differential posttranslational modifications were employed. A peptide mass tolerance equal to 30 ppm and a fragment ion mass tolerance equal to 0.8 amu were used in all searches. Monoisotopic mass type, fully trypticpeptide termini, and up to two missed cleavages were used in all searches. The SEQUEST output was filtered, validated, and analyzed using Peptide Prophet, Protein Prophet (Institute for Systems Biology, WA) and Scaffold (Proteome Software, OR) software. The balance between reliability and sensitivity of the protein identification data was set by adjusting the estimated false positive peptide identification rate (FPR) to below 0.5%. The FPR was calculated as the number of peptide matches from a “reverse” database divided by the total number of “forward” protein matches, in percentages. The semiquantitative spectral count data sets obtained for all samples were subsequently integrated and processed using the in-house written software ProMerger which allowed us to compare proteomic profiles derived from different samples and perform the downstream pathway analysis.

### Example 4

### Statistical Methods of Proteomic Analysis

Spectral counts were computed for each protein in each sample by utilizing high quality MS/MS-based peptide identifications. This example detected differentially abundant proteins between lean and obese mice, as opposed to absolute protein quantification or cross-protein comparisons of abundance, and this approach ultimately restricted attention to proteins with average spectral count (across samples) greater than 5 for better reliability. See Liu et al., 76 Anal. Chem. 4193 (2004). This obviates the need for certain within-protein normalization techniques. See Schmidt et al., 2007; Ishihama et al., 4. Mol. Cell. Proteomics 1265 (2005); Lu et al., 25 Nat. Biotech. 117 (2007). Differentially abundant proteins were identified by fit in a Poisson mixed model for each protein. Diggle et al., in ANALYSIS OF LONGITUDINAL DATA (Oxford Press, 2002). The Poisson mixed model allows for a principled treatment of discrete-count data and provides a statistically rigorous framework for the identification of differentially abundant proteins accounting for correlation among repeated measures and over-dispersion. A similar approach is followed in Choi et al. (7 Mol. Cell Proteomics 2373 (2008). This approach relied on fewer modeling assumptions than the Bayesian approach advocated by Choi et al., where variability of abundance is assumed to be constant across proteins—a strong assumption that generally does not hold in practice. The present approach does not require this assumption. Because it relies on fewer modeling assumptions, it is reasonable to expect that this procedure is, in fact, more robust to model misspecification than that of Choi et al.

The Poisson mixed model, unlike an ordinary Poisson model, accounts for over-dispersion often present in spectral count data. Indeed, a random intercept term for each mouse in the experiments was applied to account for over-dispersion. Furthermore, in order to adjust for difference in the overall protein abundance in each sample, an offset term was included depending on the total spectral counts (across all proteins) in each sample. Finally, even after including the offset term, there was a substantial differences between the experiments, thus analyses were controlled for an experiment effect. In summary, each protein fit the model described by the equation:

log(μᵢⱼₖ)=log(tᵢⱼₖ)+a+bⱼ+γₖ+δxⱼ

where μᵢⱼₖ is the expected spectral count for the i-th technical replicate from the j-th mouse in experiment k, conditional on the mean zero mouse-specific random effect bⱼ; tᵢⱼₖ is the total spectral counts in the sample; γₖ represents the k-th experiment effect; and xⱼ=0 or 1 according to whether the j-th mouse was from the lean or obese group and δ is the corresponding lean/obese effect. A total of five experiments were conducted. Each was comprised of four mice—two lean and two obese samples. In one of the experiments, two samples per mouse were available (technical replicates), while in the other four experiments only a single sample per mouse was available. Thus, for each Poisson mixed model fit, a total of 24 observations were utilized. The parameter of primary interest was δ. For each protein, a p-value was obtained corresponding to δ, and proteins were ranked by these p-values for significance, using the R library lme4 to fit the Poisson mixed models.

TABLE 1a
Up-regulated proteins in the obese liver ER proteome

| | | MW | | Fold | |
|---|---|---|---|---|---|
| Symbol | UniProt Accession | (kDa) | Nomenclature | Change | p-val |
| Acaa1b | Q8VCH0\|THIKB_MOUSE | 44 | acetyl-Coenzyme A acyltransferase 1B | 14.0 | 7.37E−12 |
| Fasn | P19096\|FAS_MOUSE | 272 | fatty acid synthase | 8.8 | 1.04E−07 |
| Oplah | Q8K010\|OPLA_MOUSE | 138 | 5-oxoprolinase (ATP-hydrolysing) | 7.0 | 1.21E−02 |
| Pcx | Q3T9S7\|Q3T9S7_MOUSE | 130 | pyruvate carboxylase | 7.0 | 4.00E−04 |
| Apoa4 | P06728\|APOA4_MOUSE | 45 | apolipoprotein A-IV | 6.0 | 1.19E−10 |
| Pklr | P53657\|KPYR_MOUSE | 62 | pyruvate kinase liver and red blood | 5.5 | 2.22E−06 |
| | | | cell | | |
| Aldh3a2 | Q5SRE0\|Q5SRE0_MOUSE | 59 | aldehyde dehydrogenase family 3, | 5.3 | 7.74E−10 |
| | | | subfamily A2 | | |
| Tuba1a | P68369\|TBA1A_MOUSE | 50 | tubulin, alpha 1A | 5.0 | 7.22E−03 |
| Tubb2b | Q9CWF2\|TBB2B_MOUSE | 50 | tubulin, beta 2B | 5.0 | 3.46E−10 |
| Gpd1 | P13707\|GPDA_MOUSE | 38 | glycerol-3-phosphate dehydrogenase 1 | 4.5 | 3.71E−04 |
| | | | (soluble) | | |
| Acaca | Q5SWU9\|COA1_MOUSE | 265 | acetyl-Coenzyme A carboxylase alpha | 4.3 | 2.01E−05 |
| Psmd1 | Q3TXS7\|PSMD1_MOUSE | 106 | proteasome (prosome, macropain) 26S | 4.0 | 2.19E−02 |
| | | | subunit, non-ATPase, 1 | | |
| Myh14 | Q6URW6\|MYH14_MOUSE | 229 | myosin, heavy polypeptide 14 | 4.0 | 9.25E−04 |
| Eno1 | P17182\|ENOA_MOUSE | 47 | enolase 1, alpha non-neuron | 4.0 | 1.70E−07 |
| Mylc2b | Q3THE2\|MLRB_MOUSE | 20 | myosin, light chain 12B, regulatory | 3.4 | 3.83E−03 |
| Ugp2 | Q91ZJ5\|UGPA_MOUSE | 57 | UDP-glucose pyrophosphorylase 2 | 3.3 | 5.47E−03 |
| Coasy | Q9DBL7\|COASY_MOUSE | 62 | Coenzyme A synthase | 3.0 | 1.05E−02 |
| Ces3 | Q8VCT4\|CES3_MOUSE | 62 | carboxylesterase 3 | 2.9 | 3.53E−03 |
| Gstm1 | A2AE89\|A2AE89_MOUSE | 24 | glutathione S-transferase, mu 1 | 2.9 | 2.27E−06 |
| Pygl | Q9ET01\|PYGL_MOUSE | 97 | liver glycogen phosphorylase | 2.7 | 3.69E−03 |
| Hbb-b1 | A8DUK7\|A8DUK7_MOUSE | 16 | hemoglobin, beta adult major chain | 2.6 | 3.52E−02 |
| Dak | Q8VC30\|DAK_MOUSE | 60 | dihydroxyacetone kinase 2 homolog | 2.6 | 1.01E−04 |
| | | | (yeast) | | |
| Fmo1 | P50285\|FMO1_MOUSE | 60 | flavin containing monooxygenase 1 | 2.5 | 1.39E−02 |
| Aldob | Q91Y97\|ALDOB_MOUSE | 40 | aldolase B, fructose-bisphosphate | 2.5 | 9.39E−08 |
| Cat | P24270\|CATA_MOUSE | 60 | catalase | 2.3 | 2.81E−02 |
| P4hb | P09103\|PDIA1_MOUSE | 57 | prolyl 4-hydroxylase, beta polypeptide | 2.1 | 1.73E−04 |
| Sds | Q8VBT2\|SDHL_MOUSE | 35 | serine dehydratase | 2.0 | 1.79E−02 |
| Gstz1 | Q9JJA0\|Q9JJA0_MOUSE | 16 | glutathione transferase zeta 1 | 2.0 | 3.45E−05 |
| | | | (maleylacetoacetate isomerase) | | |
| Ephx1 | P97869\|P97869_MOUSE | 53 | epoxide hydrolase 1, microsomal | 2.0 | 4.24E−08 |
| Maob | Q8BW75\|AOFB_MOUSE | 59 | monoamine oxidase B | 1.9 | 2.80E−06 |
| Cyb5r3 | Q9CY59\|Q9CY59_MOUSE | 34 | cytochrome b5 reductase 3 | 1.8 | 8.66E−03 |
| Trf | Q921I1\|TRFE_MOUSE | 77 | transferrin | 1.8 | 4.52E−03 |
| Cyb5 | P56395\|CYB5_MOUSE | 15 | cytochrome b-5 | 1.8 | 3.97E−02 |
| Acsl5 | Q8JZR0\|ACSL5_MOUSE | 76 | acyl-CoA synthetase long-chain family | 1.8 | 1.55E−03 |
| | | | member 5 | | |
| Phb | P67778\|PHB_MOUSE | 30 | prohibitin | 1.8 | 2.86E−02 |
| Aldh1a1 | P24549\|AL1A1_MOUSE | 54 | aldehyde dehydrogenase family 1, | 1.7 | 1.38E−03 |
| | | | subfamily A1 | | |
| Slc25a5 | P51881\|ADT2_MOUSE | 33 | solute carrier family 25 (mitochondrial | 1.7 | 6.42E−03 |
| | | | carrier, adenine nucleotide | | |
| | | | translocator), member 5 | | |
| Atp5h | Q9DCX2\|ATP5H_MOUSE | 19 | ATP synthase, H+ transporting, | 1.7 | 2.23E−02 |
| | | | mitochondrial F0 complex, subunit d | | |
| Mttp | O08601\|MTP_MOUSE | 99 | microsomal triglyceride | 1.7 | 5.71E−03 |
| | | | transfer protein | | |
| Atp5a1 | Q03265\|ATPA_MOUSE | 60 | ATP synthase, H+ transporting, | 1.7 | 1.58E−07 |
| | | | mitochondrial F1 complex, alpha | | |
| | | | subunit, isoform 1 | | |
| Fmo5 | P97872\|FMO5_MOUSE | 60 | flavin containing monooxygenase 5 | 1.6 | 3.75E−04 |
| Atp5o | Q9DB20\|ATPO_MOUSE | 23 | ATP synthase, H+ transporting, | 1.6 | 9.15E−03 |
| | | | mitochondrial F1 complex, O subunit | | |
| Etfdh | Q6PF96\|Q6PF96_MOUSE | 61 | electron transferring flavoprotein, | 1.6 | 3.01E−03 |
| | | | dehydrogenase | | |
| Mvp | Q3THX5\|Q3THX5_MOUSE | 97 | major vault protein | 1.6 | 2.57E−06 |
| Apoe | P08226\|APOE_MOUSE | 36 | apolipoprotein E | 1.6 | 2.01E−08 |
| Mat1a | Q91X83\|METK1_MOUSE | 44 | methionine adenosyltransferase I, | 1.5 | 1.95E−03 |
| | | | alpha | | |
| Gapdh | P16858\|G3P_MOUSE | 36 | glyceraldehyde-3-phosphate | 1.5 | 1.90E−02 |
| | | | dehydrogenase | | |
| Rps13 | P62301\|RS13_MOUSE | 17 | ribosomal protein S13 | 1.5 | 1.67E−02 |
| Flnb | Q80X90\|FLNB_MOUSE | 278 | filamin, beta | 1.5 | 4.31E−03 |
| Myl6 | Q60605\|MYL6_MOUSE | 17 | myosin, light polypeptide 6, alkali, | 1.5 | 5.16E−03 |
| | | | smooth muscle and non-muscle | | |

TABLE 1b
Down-regulated Proteins in the obese liver proteome

| | | MW | | Fold | |
|---|---|---|---|---|---|
| Symbol | UniProt Accession | (kDa) | Nomenclature | Change | p-val |
| Gne | A2A]63\|A2A]63_MOUSE | 11 | glucosamine | −19.0 | 3.92E−09 |
| Eif3f | Q9DCH4\|EIF3F_MOUSE | 38 | eukaryotic translation initiation | −15.5 | 5.07E−06 |
| | | | factor 3, subunit F | | |
| Eif2s2 | Q99L45\|IF2B_MOUSE | 38 | eukaryotic translation initiation | −8.5 | 5.25E−03 |
| | | | factor 2, subunit 2 (beta) | | |
| Eef1g | Q9D8N0\|EF1G_MOUSE | 50 | eukaryotic translation elongation | −8.0 | 1.08E−02 |
| | | | factor 1 gamma | | |
| Eif3g | Q9Z1D1\|EIF3G_MOUSE | 36 | eukaryotic translation initiation | −8.0 | 7.80E−04 |
| | | | factor 3, subunit G | | |
| Eif2s3x | A2AAW9\|A2AAW9_MOUSE | 37 | eukaryotic translation initiation | −7.7 | 3.06E−05 |
| | | | factor 2, subunit 3, structural gene | | |
| | | | X-linked | | |
| Egfr | Q01279\|EGFR_MOUSE | 135 | epidermal growth factor receptor | −6.5 | 2.48E−12 |
| Tdo2 | P48776\|T23O_MOUSE | 48 | tryptophan 2,3-dioxygenase | −6.0 | 3.96E−04 |
| Pfkfb1 | P70266\|F261_MOUSE | 55 | 6-phosphofructo-2-kinase/fructose- | −6.0 | 4.79E−03 |
| | | | 2,6-biphosphatase 1 | | |
| Sept9 | A2A6U3\|A2A6U3_MOUSE | 64 | septin 9 | −5.5 | 1.62E−02 |
| Eif3e | P60229\|EIF3E_MOUSE | 52 | eukaryotic translation initiation | −5.3 | 9.44E−05 |
| | | | factor 3, subunit E | | |
| Eif3m | Q3TI04\|Q3TI04_MOUSE | 43 | eukaryotic translation initiation | −5.0 | 1.63E−04 |
| | | | factor 3, subunit M | | |
| Prps1 | Q3TI27\|Q3TI27_MOUSE | 35 | phosphoribosyl pyrophosphate | −4.5 | 3.57E−02 |
| | | | synthetase 1 | | |
| Mrc1 | Q61830\|MRC1_MOUSE | 165 | mannose receptor, C type 1 | −4.4 | 2.57E−04 |
| Atp11c | Q9QZW0\|AT11C_MOUSE | 129 | ATPase, class VI, type 11C | −4.2 | 1.49E−11 |
| Gcn111 | Q3U3Z4\|Q3U3Z4_MOUSE | 118 | GCN1 general control of amino-acid | −4.0 | 1.52E−03 |
| | | | synthesis 1-like 1 (yeast) | | |
| Eif2s1 | Q6ZWX6\|IF2A_MOUSE | 36 | eukaryotic translation initiation | −3.9 | 1.07E−04 |
| | | | factor 2, subunit 1 alpha | | |
| Eif4b | Q8BGD9\|IF4B_MOUSE | 69 | eukaryotic translation initiation | −3.7 | 6.28E−04 |
| | | | factor 4B | | |
| Gstp1 | P19157\|GSTP1_MOUSE | 24 | glutathione S-transferase, pi 1 | −3.6 | 1.54E−05 |
| Eif3c | Q8R1B4\|EIF3C_MOUSE | 106 | eukaryotic translation initiation | −3.4 | 3.84E−05 |
| | | | factor 3, subunit C | | |
| Dnm2 | P39054\|DYN2_MOUSE | 98 | dynamin 2 | −3.2 | 2.97E−05 |
| Eif3h | Q8BMZ8\|Q8BMZ8_MOUSE | 7 | eukaryotic translation initiation | −3.1 | 5.95E−05 |
| | | | factor 3, subunit H | | |
| Eif3i | Q9QZD9\|EIF3I_MOUSE | 36 | eukaryotic translation initiation | −3.1 | 3.24E−03 |
| | | | factor 3, subunit I | | |
| Eif3d | O70194\|EIF3D_MOUSE | 64 | eukaryotic translation initiation | −3.1 | 4.08E−05 |
| | | | factor 3, subunit D | | |
| Eif3b | Q8CI]3\|Q8CI]3_MOUSE | 109 | eukaryotic translation initiation | −3.1 | 1.50E−09 |
| | | | factor 3, subunit B | | |
| Actr1b | Q8R5C5\|ACTY_MOUSE | 42 | ARP1 actin-related protein 1 | −3.0 | 1.89E−02 |
| | | | homolog B, centractin beta (yeast) | | |
| Cad | Q6P9L1\|Q6P9L1_MOUSE | 158 | carbamoyl-phosphate synthetase 2, | −3.0 | 9.82E−04 |
| | | | aspartate transcarbamylase, | | |
| | | | and dihydroorotase | | |
| Abce1 | P61222\|ABCE1_MOUSE | 67 | ATP-binding cassette, sub-family E | −2.8 | 1.00E−04 |
| | | | (OABP), member 1 | | |
| Eif3eip | Q8QZY1\|IF3EI_MOUSE | 67 | eukaryotic translation initiation | −2.8 | 4.61E−10 |
| | | | factor 3, subunit L | | |
| Lman1 | Q3U944\|Q3U944_MOUSE | 61 | lectin, mannose-binding, 1 | −2.8 | 4.30E−02 |
| Asgr1 | P34927\|ASGR1_MOUSE | 33 | asialoglycoprotein receptor 1 | −2.7 | 9.43E−14 |
| Lrp1 | Q91ZX7\|LRP1_MOUSE | 505 | low density lipoprotein receptor- | −2.7 | 5.92E−09 |
| | | | related protein 1 | | |
| Usp9x | A2AD18\|A2AD18_MOUSE | 291 | ubiquitin specific peptidase 9, | −2.7 | 3.96E−02 |
| | | | X chromosome | | |
| Eif3a | P23116\|EIF3A_MOUSE | 162 | eukaryotic translation initiation factor | −2.7 | 8.68E−09 |
| | | | 3, subunit A | | |
| Scamp3 | Q3TDM8\|Q3TDM8_MOUSE | 35 | secretory carrier membrane protein 3 | −2.6 | 3.64E−10 |
| Rps8 | P62242\|RS8_MOUSE | 24 | ribosomal protein S8 | −2.5 | 2.38E−04 |
| Cyp2c50 | Q91X77\|CY250_MOUSE | 56 | cytochrome P450, family 2, subfamily | −2.5 | 6.51E−03 |
| | | | c, polypeptide 50 | | |
| Rrbp1 | A2AV]7\|A2AV]7_MOUSE | 158 | ribosome binding protein 1 | −2.5 | 5.84E−03 |
| Eif3j | Q3UGC7\|Q3UGC7_MOUSE | 29 | eukaryotic translation initiation | −2.4 | 8.60E−05 |
| | | | factor 3, subunit J | | |
| Hpx | Q3UKP2\|Q3UKP2_MOUSE | 51 | hemopexin | −2.4 | 6.30E−04 |
| Atl2 | Q6PA06\|ATLA2_MOUSE | 66 | atlastin GTPase 2 | −2.2 | 3.28E−03 |
| Cyp2d9 | P11714\|CP2D9_MOUSE | 57 | cytochrome P450, family 2, | −2.2 | 3.68E−02 |
| | | | subfamily d, polypeptide 9 | | |
| Copb1 | Q9]IF7\|COPB_MOUSE | 107 | coatomer protein complex, | −2.2 | 1.05E−03 |
| | | | subunit beta 1 | | |
| Vps26a | P40336\|VP26A_MOUSE | 38 | vacuolar protein sorting 26 homolog A | −2.2 | 1.11E−04 |
| | | | (yeast) | | |
| Ccdc22 | Q9]IG7\|CCD22_MOUSE | 71 | coiled-coil domain containing 22 | −2.2 | 2.23E−03 |
| Ugt2b1 | Q8R084\|Q8R084_MOUSE | 60 | UDP glucuronosyltransferase 2 family, | −2.2 | 1.16E−03 |
| | | | polypeptide B1 | | |
| Copa | Q8BTF0\|Q8BTF0_MOUSE | 139 | coatomer protein complex | −2.1 | 1.98E−04 |
| | | | subunit alpha | | |
| Pigr | O70570\|PIGR_MOUSE | 85 | polymeric immunoglobulin receptor | −2.1 | 1.60E−10 |
| Cyp1a2 | P00186\|CP1A2_MOUSE | 58 | cytochrome P450, family 1, | −2.1 | 2.53E−03 |
| | | | subfamily a, polypeptide 2 | | |
| Cct3 | P80318\|TCPG_MOUSE | 61 | chaperonin containing Tcp1, subunit 3 | −2.0 | 1.96E−02 |
| | | | (gamma) | | |
| Gnb2l1 | P68040\|GBLP_MOUSE | 35 | guanine nucleotide binding protein | −2.0 | 1.41E−02 |
| | | | (G protein), beta polypeptide 2 like 1 | | |
| Dpp4 | P28843\|DPP4_MOUSE | 87 | dipeptidylpeptidase 4 | −2.0 | 5.62E−03 |
| Mup12 | A2CEK7\|A2CEK7_MOUSE | 21 | major urinary protein 12 | −2.0 | 1.01E−02 |
| Hp | Q61646\|HPT_MOUSE | 39 | haptoglobin | −2.0 | 4.50E−04 |
| M6pr | P24668\|MPRD_MOUSE | 31 | mannose-6-phosphate receptor, | −2.0 | 3.18E−03 |
| | | | cation dependent | | |
| Ap1m1 | P35585\|AP1M1_MOUSE | 49 | adaptor-related protein complex AP-1 | −2.0 | 2.44E−03 |
| | | | mu subunit 1 | | |
| Eif4a1 | P60843\|IF4A1_MOUSE | 46 | eukaryotic translation initiation | −2.0 | 5.05E−03 |
| | | | factor 4A1 | | |
| Abca6 | Q8K441\|ABCA6_MOUSE | 183 | ATP-binding cassette, sub-family A | −1.8 | 6.82E−03 |
| | | | (ABC1), member 6 | | |
| Anxa11 | P97384\|ANX11_MOUSE | 54 | annexin A11 | −1.8 | 2.23E−02 |
| Igf2r | Q07113\|MPRI_MOUSE | 274 | insulin-like growth factor 2 receptor | −1.8 | 7.61E−04 |
| Cpne3 | Q8BT60\|CPNE3_MOUSE | 60 | copine III | −1.8 | 1.79E−10 |
| Vps35 | Q9EQH3\|VPS35_MOUSE | 92 | vacuolar protein sorting 35 | −1.7 | 6.09E−04 |
| Clint1 | Q3UGL3\|Q3UGL3_MOUSE | 68 | clathrin interactor 1 | −1.7 | 2.82E−04 |
| Cope | O89079\|COPE_MOUSE | 35 | coatomer protein complex, | −1.7 | 1.11E−02 |
| | | | subunit epsilon | | |
| Dnaja1 | P63037\|DN]A1_MOUSE | 45 | Dna] (Hsp40) homolog, subfamily A, | −1.6 | 1.66E−03 |
| | | | member 1 | | |
| Rps6 | P62754\|RS6_MOUSE | 29 | ribosomal protein S6 | −1.6 | 1.29E−04 |
| Rdh7 | O88451\|RDH7_MOUSE | 36 | retinol dehydrogenase 7 | −1.6 | 2.30E−05 |
| Arcn1 | Q3U4S9\|Q3U4S9_MOUSE | 57 | archain 1 | −1.5 | 2.85E−02 |
| Aadac | Q99PG0\|AAAD_MOUSE | 45 | arylacetamide deacetylase (esterase) | −1.5 | 2.90E−02 |
| Ugt2b5 | P17717\|UD2B5 MOUSE | 61 | UDP glucuronosyltransferase 2 family, | −1.5 | 5.89E−03 |
| | | | polypeptide B5 | | |

### Example 5

### Bioinformatic Analysis of Proteomics

Proteins identified as significantly up- or down-regulated in the obese ER proteome were analyzed by Database for Annotation, Visualization and Integrated Discovery (DAVID, available on the internet at the ncifcrf site (see Dennis et al., 4 Genome Biol. P3 (2003); Huang et al., 4 Nat. Protoc. 44 (2009)), as plotted in R. Clustering analysis was carried out with the Cluster3.0 program (Eisen et al., 95 PNAS 14863 (1998)), and visualized either in JavaTreeview or MeV (Id.; Saeed et al., 411 Meths. Enzymol. 134 (2006)). Functional annotation charts of proteins of interest (absolute median fold change ˜1.5, significance of fold change ˜0.05, average unadjusted spectral count of 5 across all experiments) were generated using the ‘Biological Pathways’ subset of Gene Ontology included in the DAVID System using all identified ER proteins as the background set. Biological pathway annotations were manually curated to remove redundant (identical) annotations associated with the same sets of proteins.

### Example 6

### Quantitative Profiling of Lipids and Fatty Acid Compositions of ER and Statistics

ER pellets (˜50 mg) were resuspended in 1 ml of 0.25 M sucrose, 200 μl of which was used for lipid extraction in the presence of authentic internal standards by the method of Folch et al., with chloroform:methanol (2:1 v/v). See Folch et al., 226 J. Biol. Chem. 497 (1957). Individual lipid classes were separated and quantified by liquid chromatography (Agilent Technologies model 1100 Series). To obtain the quantitative composition of fatty acids for each lipid class, the separated lipids were transesterified in 1% sulfuric acid/methanol at 100° C. for 45 minutes and extracted by 0.05% butylated hydroxytoluene/hexane. The resulting fatty acid methyl esters were quantified by gas chromatography (Agilent Technologies model 6890) under nitrogen.

The nmol % of each fatty acid was computed as the nmole quantity of the individual fatty acid divided by the total nmole amount of fatty acid isolated from each lipid class of each ER sample. The nmole % profile of fatty acids was then averaged in all six lean ER samples to examine the differences in the fatty acid profile that existed among different lipid classes. To identify compositional differences between control and experimental groups, Student's t-tests were performed for all fatty acid/lipid class combinations (26×9). The mean difference of nmol % for each fatty acid/lipid class combination with p<0.05 were visualized in MeV34. Complete cluster analyses were performed for the fatty acid compositions of control and experimental groups using the Cluster3.0 program33 with the following filter setting: 100% present, at least 50% samples with nmole %˜2 and (max-min) ˜1.

TABLE 2
Lipid composition of ER prepared from obese and lean mouse liver tissues

| Lipid Class | obese mouse | | | | | | lean mouse | | |
|---|---|---|---|---|---|---|---|---|---|
| (nmol %) | #1 | #2 | #3 | #4 | #5 | #6 | #1 | #2 | #3 |
| Cholesterol | 4.071 | 8.442 | 1.183 | 1.691 | 2.442 | 1.381 | 1.488 | 3.490 | 6.031 |
| Ester | | | | | | | | | |
| Diacyl- | 5.617 | 3.476 | 1.982 | 3.470 | 4.269 | 1.968 | 1A54 | 2557 | 3577 |
| glycerol | | | | | | | | | |
| Free | 17.245 | 18.169 | 10.058 | 15.928 | 21.385 | 7.233 | 9.494 | 9.155 | 13.603 |
| cholesterol | | | | | | | | | |
| Free fatty acid | 8.286 | 10.921 | 5.763 | 15.017 | 5.356 | 8.776 | 5.295 | 8.452 | 20.915 |
| Triacyl- | 12.320 | 9.441 | 11.24 | 6.651 | 11386 | 9.335 | 9.986 | 19.135 | 5.945 |
| glycerol | | | | | | | | | |
| Phospholipids | 52.461 | 48.849 | 69.767 | 62.251 | 55.161 | 71.308 | 72.283 | 66.212 | 49.980 |
| Cardiolipin | 4.994 | 3.331 | 2.680 | 2.974 | 3.261 | 2.731 | 4.543 | 3.237 | 4.469 |
| Lysophospha- | 3.272 | 4.599 | 1.776 | 3.366 | 3.960 | 2.014 | 2.923 | 1.488 | 3.953 |
| tidylcholine | | | | | | | | | |
| Phosphatidyl- | 26.088 | 21.077 | 36.680 | 36.270 | 26.469 | 41.815 | 31.917 | 33.044 | 20.967 |
| choline | | | | | | | | | |
| Phosphatidyl- | 12.230 | 12.416 | 22.146 | 13.846 | 16.414 | 20.947 | 27.103 | 24159 | 13.067 |
| ethanolamine | | | | | | | | | |
| Phosphatidyl- | 5.876 | 0.426 | 4.485 | 5.795 | 503 | 3.920 | 5.491 | 4.284 | 7.424 |
| serine | | | | | | | | | |
| PC/PE | 2.133 | 1.698 | 1.747 | 2.620 | 1.610 | 1.996 | 1.164 | 1.368 | 1.695 |
| PC/PS | 2.081 | 1.672 | 4.938 | 2.389 | 3.273 | 5.483 | 4.992 | 5.639 | 1.748 |

| Lipid Class | lean mouse | | | diet # | | ob. | lean | T |
|---|---|---|---|---|---|---|---|---|
| (nmol %) | #4 | #5 | #6 | 1 | 2 | ave | ave | TEST |
| Cholesterol | 5183 | 4.367 | 1.354 | 0.010 | 0.010 | 3.201 | 3.769 | 0.696 |
| Ester | | | | | | | | |
| Diacyl- | 4 268 | 1.442 | 2.468 | 0.038 | 0.037 | 3.464 | 2.628 | 0.281 |
| glycerol | | | | | | | | |
| Free | 18.338 | 11.266 | 9528 | 0.118 | 0.121 | 15.118 | 11.897 | 0.251 |
| cholesterol | | | | | | | | |
| Free fatty acid | 12.751 | 6.277 | 7.163 | 0.516 | 0.005 | 8.187 | 10.133 | 0.466 |
| Triacyl- | 7.998 | 9 542 | 11.1 | 0.733 | 9.732 | 10.561 | 9.176 | 7.45 |
| glycerol | | | | | | | | |
| Phospholipids | 50.813 | 67.056 | 68.065 | 0.995 | 0.995 | 59.966 | 62.393 | 0.665 |
| Cardiolipin | 3.471 | 3.590 | 3.779 | 0.032 | 6.031 | 1.323 | 3.848 | 0.237 |
| Lysophospha- | 6.940 | 2.102 | 1.529 | 0.011 | 0.011 | 3.164 | 3.156 | 0.993 |
| tidylcholine | | | | | | | | |
| Phosphatidyl- | 17.553 | 31.225 | 32 349 | 0.027 | 0.026 | 31.733 | 27.841 | 0.394 |
| choline | | | | | | | | |
| Phosphatidyl- | 15.011 | 4.292 | 25.826 | 0.019 | 0.021 | 16.338 | 21.794 | 0.105 |
| ethanolamine | | | | | | | | |
| Phosphatidyl- | 7.888 | 4.247 | 4.591 | 5.556 | 0.046 | 5.404 | 5.754 | 0.675 |
| serine | | | | | | | | |
| PC/PE | 1.169 | 1.235 | 1.252 | 1.389 | 1.229 | 1.967 | 1.299 | 0.003 |
| PC/PS | 1.915 | 5.218 | 5.624 | 4.195 | 4.446 | 3.3136 | 4.189 | 0.393 |

### Example 7

### Calcium Transport Assays

The calcium transport assay for measuring Serca activity was adapted from Moore et al. (250 J. Biol. Chem. 4562 (1975)). Briefly, fresh liver tissues were homogenized in 10 volumes of buffer containing 0.25 M sucrose, 2 mM Tris pH7.4 and 1 mM DTT and EDTA-free protease inhibitor. The ER pellet was obtained after a series of centrifugation as described in the previous section, and then resuspended in 0.25 M sucrose. The same procedure was employed to isolate microsomes from cultured Hepa1-6 cells except that cell pellet was lysed in hypotonic 0.1 M sucrose, 2 mM Tris pH7.4, 1 mM DTT and EDTA-free protease inhibitor. The calcium transport assay was carried out in reaction buffer containing 0.1 M KCl, 30 mM, 5 mM NaN₃, 5 mM MgCl₂, 5 mM K₂C₂O₄, 501&M of CaCl₂ (plus 1 μCi/μmol of ⁴⁵Ca), 1 μM Rethenium Red, 5 mM ATP. The reaction was started by the addition of microsomes containing 150 μg proteins for 15 min in a 37° C. water bath and stopped by the addition of 0.15 M KCl, 1 mM LaCl₃ and filtered through a 0.2μ HT Tuffryn membrane (PALL Corporation, NY). The calcium transport experiment with lipid overloading was carried out essentially as previously described (Li et al., 2004) except that liposomes were made of egg derived PC and PE by the ethanol injection method (Watanabe et al., 45 J. Electron. Mocrosc. 171 (1996)). The amount of SERCA independent calcium transport was quantified in the presence of 10 μM thapsigargin and subtracted from the calculation.

### Example 8

### Western Blotting, Real-Time Quantitative PCR and Molecular Cloning

For the preparation of total cellular proteins, ˜0.1 g of liver tissues were homogenized in 1 ml of a cold lysis buffer containing 50 mM Tris-HCl (pH 7.0), 2 mM EGTA, 5 mM EDTA, 30 mM NaF, 10 mM Na3VO4, 10 mM Na4P2O7, 40 mM 3-glycerophosphate, 1% NP-40, and 1% protease inhibitor cocktail. After a brief centrifugation (200 g×10 min) to pellet down cell debris, ⅕ volume of 6× Laemmli buffer was added into the whole cell lysate, boiled and centrifuged at 10,000 g for 10 min. Protein concentrations were quantified with Bio-Rad Dc Protein Assay (Bio-Rad, CA). Western blotting of protein of interest was done as previously described. Erbay et al., 2009; Ozcan et al., 2006. Total RNA was extracted with Trizol reagent according to manufacturer's recommendations. A total of 2 μg of RNA was used for cDNA synthesis using High Capacity cDNA archiving system (Applied Biosystems). The SYBR real-time PCR system was used to quantify the transcript abundance for genes of interest (Table S6). Either 18S or 28S rRNA was used for internal control.

### Example 9

### Adenovirus-Mediated Loss- or Gain-of-Function Experiments

For Pemt knockdown experiments, a series of DNA hairpins specifically targeting the mouse Pemt gene were designed by RNAxs (see Tafer et al., 26 Nat. Biotech. 578 (2008)), synthesized, cloned into the pENTR/U6 system (Invitrogen, CA) and tested in the Hepa1-6 cell line. The sequence with best efficacy, and it has 5nt mismatch with the next closest match of genes, were recloned into the pAD/Block-iT-DEST system through recombination, as described. Cao et al., 134 Cell 933 (2008). The LacZ shRNA was also cloned into the pAD/Block-iT-DEST system as control. For Serca2b over-expression experiment, the open reading frame of human Serca2b or Gfp (control) was amplified, cloned into pENTR/TOPO vector and then recombined into the pAD/CMV/V5-DEST vector. Adenovirus (serotype 5, Ad5) for the construct of interest was produced and amplified in 293A cells, purified using CsCl column, desalted, and 1×10¹¹ virus particles were used for each injection. Adenovirus transductions of mice were performed between 10-11 weeks of age. Blood glucose levels were measured after 6 hr of food withdrawal (9 am-3 pm) at before and 5 days post-injection and at the time of harvest (9-12 days). For histological analysis, liver tissues were fixed in 10% formalin solution, and sectioned for Hematoxylin and Eosin staining. All oligonucleotide sequences are listed in Table 5.

TABLE 3

| | Samples | | | | | | | | | | |
|---|---|---|---|---|---|---|---|---|---|---|---|
| | Ob/pemt: shRNAi | | | | ob/lacZ RNAi | | | | pemt. | lacZ. | |
| Lipid Class (nmol %) | 1 | 2 | 3 | 4 | 1 | 2 | 3 | 4 | ave | ave | TTEST |
| Cholesterol Ester | 6.39 | 1.95 | 5.11 | 2.55 | 1.43 | 2.22 | 1.43 | 3.98 | 4.00 | 2.27 | 0.2017 |
| Diacylglycerol | 1.17 | 1.45 | 1.51 | 1.45 | 1.79 | 2.60 | 1.46 | 1.53 | 1.40 | 1.85 | 0.1515 |
| Free cholesterol | 6.96 | 7.83 | 7.76 | 6.84 | 11.42 | 7.99 | 6.85 | 4.01 | 7.35 | 7.57 | 0.8909 |
| Free fatty acid | 2.67 | 2.59 | 3.91 | 3.47 | 3.37 | 2.66 | 2.36 | 2.26 | 3.16 | 2.66 | 0.2641 |
| Triacylglycerol | 11.69 | 8.57 | 9.33 | 13.46 | 12.33 | 11.45 | 19.90 | 19.76 | 10.76 | 15.86 | 0.0933 |
| Phospholipids | 71.13 | 77.61 | 72.36 | 72.22 | 69.67 | 73.07 | 67.98 | 68.46 | 73.33 | 69.79 | 0.1047 |
| Cardiolipin | 9.99 | 10.12 | 7.67 | 8.47 | 9.26 | 6.85 | 7.54 | 7.73 | 9.06 | 7.84 | 0.1712 |
| Lysophosphatidylcholine | 1.77 | 1.37 | 1.09 | 1.25 | 1.28 | 1.23 | 1.15 | 1.28 | 1.37 | 1.23 | 0.3917 |
| Phosphatidylcholine | 30.07 | 34.20 | 32.59 | 35.16 | 37.46 | 41.03 | 37.96 | 40.60 | 33.00 | 39.26 | 0.0047 |
| Phosphatidylethanolamine | 24.11 | 25.46 | 24.55 | 21.71 | 18.09 | 17.73 | 17.27 | 15.65 | 23.95 | 17.18 | 0.0004 |
| Phosphatidylserine | 5.19 | 6.46 | 6.48 | 5.64 | 3.59 | 6.22 | 4.07 | 3.21 | 5.94 | 4.27 | 0.0660 |
| PC/PE | 1.25 | 1.34 | 1.33 | 1.62 | 2.07 | 2.31 | 2.20 | 2.60 | 1.38 | 2.29 | 0.0006 |
| PC/PS | 5.79 | 5.29 | 5.04 | 6.23 | 10.45 | 6.60 | 9.34 | 12.65 | 5.59 | 9.76 | 0.0176 |

TABLE 4

| | Fatty Acids | | | | | | | | | | | | |
|---|---|---|---|---|---|---|---|---|---|---|---|---|---|
| | (mol %) | 14:0 | 15:0 | 16:0 | 18:0 | 20:0 | 22:0 | 14:1n5 | 16:1n7 | 18:1n7 | 18:1n9 | 20:1n9 | 20:3n9 |
| [symbol] | Cardiolpin | −0.376 | NS | NS | 2.764 | NS | NS | NS | NS | NS | NS | NS | NS |
| | Cholesterol | [symbol] .206 | NS | NS | NS | NS | NS | NS | NS | NS | NS | NS | NS |
| | Ester | | | | | | | | | | | | |
| | Diacyl- | 0.5 [symbol] | NS | 4.832 | 5 [symbol] | NS | NS | NS | −1.519 | NS | −5. [symbol] | NS | NS |
| | glycerol | | | | | | | | | | | | |
| | Free fatty | 1. [symbol] | NS | NS | NS | NS | NS | 0.183 | NS | [symbol] | NS | NS | NS |
| | acid | | | | | | | | | | | | |
| | Lysophospha- | 0.442 | NS | NS | NS | NS | NS | NS | NS | NS | NS | NS | −0.197 |
| | tidylcholine | | | | | | | | | | | | |
| | Phosphatidyl- | 0.09 | 0.042 | 5.739 | [symbol] | NS | NS | NS | 0.427 | NS | NS | −0.0 [symbol] | 0.0 [symbol] |
| | choline | | | | | | | | | | | | |
| | Phosphatidyl- | −0.131 | NS | 2.482 | −2. [symbol] | NS | −0.0 [symbol] | NS | NS | NS | NS | NS | NS |
| | ethanolamine | | | | | | | | | | | | |
| | Phosphatidyl- | 0.281 | NS | NS | [symbol] .075 | [symbol] | NS | NS | NS | NS | NS | NS | NS |
| | serine | | | | | | | | | | | | |
| | Triacyl- | 0. [symbol] | NS | NS | NS | NS | NS | NS | NS | [symbol] | NS | −0. [symbol] | NS |
| | glycerol | | | | | | | | | | | | |

| | Fatty Acids | | | | | | | | | | % |
|---|---|---|---|---|---|---|---|---|---|---|---|
| | (mol %) | 18:2n6 | 18:3n6 | 20:2n6 | 20:3n6 | 20:4n6 | 22:4n6 | 20:5n3 | 22: [symbol] n3 | 22: [symbol] | [symbol] FA |
| [symbol] | Cardiolpin | [symbol] | NS | NS | NS | −0.149 | NS | NS | NS | 4.4 [symbol] | NS |
| | Cholesterol | NS | NS | NS | NS | NS | NS | NS | NS | NS | NS |
| | Ester | | | | | | | | | | |
| | Diacyl- | NS | NS | NS | NS | NS | NS | NS | NS | −2.148 | 11.77 |
| | glycerol | | | | | | | | | | |
| | Free fatty | NS | NS | NS | NS | NS | NS | NS | NS | NS | NS |
| | acid | | | | | | | | | | |
| | Lysophospha- | NS | NS | NS | NS | NS | NS | NS | NS | NS | NS |
| | tidylcholine | | | | | | | | | | |
| | Phosphatidyl- | 2.524 | NS | −0.0 [symbol] | NS | NS | NS | NS | NS | −5.9 [symbol] | 1.979 |
| | choline | | | | | | | | | | |
| | Phosphatidyl- | 1.81 [symbol] | NS | NS | NS | [symbol] | NS | NS | 0.48 [symbol] | 4.345 | NS |
| | ethanolamine | | | | | | | | | | |
| | Phosphatidyl- | 0. [symbol] | NS | NS | NS | 4.518 | NS | NS | NS | 2.27 [symbol] | NS |
| | serine | | | | | | | | | | |
| | Triacyl- | NS | 0.0 [symbol] | NS | 0.16 | 0. [symbol] | [symbol] | [symbol] | NS | 0. [symbol] | [symbol] |
| | glycerol | | | | | | | | | | |

| | Fatty Acids | % | % | % | % | % | % |
|---|---|---|---|---|---|---|---|
| | (mol %) | [symbol] UFA | PUFA | n3 | n6 | n7 | n9 |
| [symbol] | Cardiolpin | NS | 3.353 | NS | −1.804 | NS | NS |
| | Cholesterol | NS | NS | NS | NS | NS | NS |
| | Ester | | | | | | |
| | Diacyl- | −7.932 | NS | NS | NS | −2.582 | −5.362 |
| | glycerol | | | | | | |
| | Free fatty | NS | NS | NS | NS | −1.004 | NS |
| | acid | | | | | | |
| | Lysophospha- | NS | NS | NS | NS | NS | NS |
| | tidylcholine | | | | | | |
| | Phosphatidyl- | NS | −4.874 | −6.089 | NS | NS | NS |
| | choline | | | | | | |
| | Phosphatidyl- | NS | NS | 5.219 | −4.5 [symbol] | NS | NS |
| | ethanolamine | | | | | | |
| | Phosphatidyl- | NS | NS | NS | −4.121 | −0.469 | NS |
| | serine | | | | | | |
| | Triacyl- | [symbol] | NS | NS | NS | NS | −5.5 [symbol] |
| | glycerol | | | | | | |

*NS: no significant changes ( [symbol] student t-test) [symbol] Values are [symbol] (mol%) difference between [symbol] [symbol]
indicates data missing or illegible when filed

TABLE 5

| Genes | Orientation | Sequence | Usage |
|---|---|---|---|
| 18S | forward | AGCCCCTGCCCTTTGTACACA | q-PCR |
| 18S | reverse | CGATCCGAGGGCCTCACTA | q-PCR |
| 28S | forward | TGTTGACGCGATGTGATTTCTGCC | q-PCR |
| 28S | reverse | AGATGACGAGGCATTTGGCTACCT | q-PCR |
| Ces3 | forward | ATGCGCCTCTACCCTCTGATA | q-PCR |
| Ces3 | reverse | AGCAAATCTCAAGGAGCCAAG | q-PCR |
| Dak | forward | TCGGGAAAGGGATGCTAACAG | q-PCR |
| Dak | reverse | CAAGTCCAAAGTTGAGCCGAT | q-PCR |
| Dgat2 | forward | GCGCTACTTCCGAGACTACTT | q-PCR |
| Dgat2 | reverse | GGGCCTTATGCCAGGAAACT | q-PCR |
| Fas | forward | TATCAAGGAGGCCCATTTTGC | q-PCR |
| Fas | reverse | TGTTTCCACTTCTAAACCATGCT | q-PCR |
| Herpud1 | forward | CTGGGGACTCCTCAAGTGATG | q-PCR |
| Herpud1 | reverse | ACGTTGTGTAGCCAGAGAAGC | q-PCR |
| Lac2 | top | CACCGCTACACAAATCAGCGATTTCGAAAAATCGCTGATTTGTGTAG | shRNA |
| Lac2 | bottom | AAAACTACACAAATCAGCGATTTTTCGAAATCGCTGATTTGTGTAGC | shRNA |
| Mttp | forward | ATACAAGCTCACGTACTCCACT | q-PCR |
| Mttp | reverse | TCCACAGTAACACAACGTCCA | q-PCR |
| Pcyt1a | forward | GATGCACAGAGTTCAGCTAAAGT | q-PCR |
| Pcyt1a | reverse | TGGCTGCCGTAAACCAACTG | q-PCR |
| Pcyt2 | forward | TGTGTTCACGGCAATGACATC | q-PCR |
| Pcyt2 | reverse | TTCCCGGTACTCAGAGGACAT | q-PCR |
| Pemt | forward | TTGGGGATTCGTGTTTGTGCT | q-PCR |
| Pemt | reverse | CACGCTGAAGGGAAATGTGG | q-PCR |
| Ptdss1 | forward | GCAGGACTCTGAGCAAGGATG | q-PCR |
| Ptdss1 | reverse | GGCGAAGTACATGAGGCTGAT | q-PCR |
| Ptdss2 | forward | GGATTGCCTTTCAGTTCACGC | q-PCR |
| Ptdss2 | reverse | AGGTAGAAGGTGTTCAGCTCTG | q-PCR |
| Scd1 | forward | TTCTTGCGATACACTCTGGTGC | q-PCR |
| Scd1 | reverse | CGGGATTGAATGTTCTTGTCGT | q-PCR |
| Serca2 | forward | CATGCACCGATGGGATTTCCT | q-PCR |
| (Atp2a2) | | | |
| Serca2 | reverse | CGCTAAAGTTAGTGTCTGTGCT | q-PCR |
| (Atp2a2) | | | |
| Pemt | top | CACCGCCATGTCCCGACACACTAACTCGAGTTAGTGTGTCGGGACATGG | shRNA |
| Pemt | bottom | AAAACCATGTCCCGACACACTAACTCGAGTTAGTGTGTCGGGACATGGC | shRNA |
| Serca2b | forward | CACCGCCGTTTGTAATTCTGCTTATCTCGAGATAAGCAGAATTACAAACGGC | shRNA |
| Serca2b | reverse | AAAAAGCCGTTTGTAATTCTGCTTATCTCGAGATAAGCAGAATTACAAACGGC | shRNA |
| Serca2b | forward | GCCATGGAGAACGCGCACAC | ORF cloning |
| Serca2b | reverse | AGACCAGAACATATCGCTAAAGTTAG | ORF cloning |

1. A method of treating hepatic chronic endoplasmic reticulum (ER) stress in an obese subject comprising modulating the phosphatidylcholine/phosphatidylethanolamine (PC/PE) ratio in the liver, wherein the subject is suffering from type 2 diabetes, dislipidemia, fatty liver disease, inflammation, or atherosclerosis; and wherein the correcting improves glucose homeostasis.

2. The method of claim 1, wherein modulating is lowering the PC/PE ratio to about 1.3

3. The method of claim 1, wherein the modulating comprises genetic, chemical or dietary intervention.

4. The method of claim 3, comprising inhibiting expression or function of phosphatidylethanolamine N-methyltransferase, encoded by Pemt.

5. A method of treating hepatic chronic endoplasmic reticulum (ER) stress in an obese subject comprising modulating calcium homeostasis in the liver, wherein the subject is suffering from type 2 diabetes, lipodemia, fatty liver disease, inflammation, or atherosclerosis; and wherein the correcting improves glucose homeostasis.

6. The method of claim 5, wherein the modulating comprises genetic, chemical or dietary intervention.

7. The method of claim 5, wherein the modulating comprises increasing hepatic concentration, expression or activity of sarco/endoplasmic reticulum calcium ATPase (SERCA).

8. The method of claim 1, wherein the modulating comprises inhibiting de novo synthesis of saturated fatty acids and monounsaturated fatty acids in liver.

9. The method of claim 1, further comprising the step of monitoring expression of asialoglycoprotein receptor (ASGR) and/or haptoglobin (HP).

10. The method of claim 1, wherein said modulating comprises down-regulating hepatic expression of at least one of:
a de novo lipogenesis gene selected from Fas, Scd1, Ces3, Dgat2 and Dak2;
a phospholipid synthesis gene selected from Pcyt1a and Pemt;
a lipoprotein synthesis gene ApoA4; or
a gene involved in glucose production selected from G6 and Pck1.


SEQUENCE LISTING (38 sequences)

SEQ ID NO: 1 - DNA, 21 bp, Artificial Sequence
  Description of Artificial Sequence Synthetic primer
agcccctgccctttgtacaca

SEQ ID NO: 2 - DNA, 19 bp, Artificial Sequence
  Description of Artificial Sequence Synthetic primer
cgatccgagggcctcacta

SEQ ID NO: 3 - DNA, 24 bp, Artificial Sequence
  Description of Artificial Sequence Synthetic primer
tgttgacgcgatgtgatttctgcc

SEQ ID NO: 4 - DNA, 24 bp, Artificial Sequence
  Description of Artificial Sequence Synthetic primer
agatgacgaggcatttggctacct

SEQ ID NO: 5 - DNA, 21 bp, Artificial Sequence
  Description of Artificial Sequence Synthetic primer
atgcgcctctaccctctgata

SEQ ID NO: 6 - DNA, 21 bp, Artificial Sequence
  Description of Artificial Sequence Synthetic primer
agcaaatctcaaggagccaag

SEQ ID NO: 7 - DNA, 21 bp, Artificial Sequence
  Description of Artificial Sequence Synthetic primer
tcgggaaagggatgctaacag

SEQ ID NO: 8 - DNA, 21 bp, Artificial Sequence
  Description of Artificial Sequence Synthetic primer
caagtccaaagttgagccgat

SEQ ID NO: 9 - DNA, 21 bp, Artificial Sequence
  Description of Artificial Sequence Synthetic primer
gcgctacttccgagactactt

SEQ ID NO: 10 - DNA, 20 bp, Artificial Sequence
  Description of Artificial Sequence Synthetic primer
gggccttatgccaggaaact

SEQ ID NO: 11 - DNA, 21 bp, Artificial Sequence
  Description of Artificial Sequence Synthetic primer
tatcaaggaggcccattttgc

SEQ ID NO: 12 - DNA, 23 bp, Artificial Sequence
  Description of Artificial Sequence Synthetic primer
tgtttccacttctaaaccatgct

SEQ ID NO: 13 - DNA, 21 bp, Artificial Sequence
  Description of Artificial Sequence Synthetic primer
ctggggactcctcaagtgatg

SEQ ID NO: 14 - DNA, 21 bp, Artificial Sequence
  Description of Artificial Sequence Synthetic primer
acgttgtgtagccagagaagc

SEQ ID NO: 15 - DNA, 47 bp, Artificial Sequence
  Description of Artificial Sequence Synthetic primer
caccgctacacaaatcagcgatttcgaaaaatcgctgatttgtgtag

SEQ ID NO: 16 - DNA, 47 bp, Artificial Sequence
  Description of Artificial Sequence Synthetic primer
aaaactacacaaatcagcgatttttcgaaatcgctgatttgtgtagc

SEQ ID NO: 17 - DNA, 22 bp, Artificial Sequence
  Description of Artificial Sequence Synthetic primer
atacaagctcacgtactccact

SEQ ID NO: 18 - DNA, 21 bp, Artificial Sequence
  Description of Artificial Sequence Synthetic primer
tccacagtaacacaacgtcca

SEQ ID NO: 19 - DNA, 23 bp, Artificial Sequence
  Description of Artificial Sequence Synthetic primer
gatgcacagagttcagctaaagt

SEQ ID NO: 20 - DNA, 20 bp, Artificial Sequence
  Description of Artificial Sequence Synthetic primer
tggctgccgtaaaccaactg

SEQ ID NO: 21 - DNA, 21 bp, Artificial Sequence
  Description of Artificial Sequence Synthetic primer
tgtgttcacggcaatgacatc

SEQ ID NO: 22 - DNA, 21 bp, Artificial Sequence
  Description of Artificial Sequence Synthetic primer
ttcccggtactcagaggacat

SEQ ID NO: 23 - DNA, 21 bp, Artificial Sequence
  Description of Artificial Sequence Synthetic primer
ttggggattcgtgtttgtgct

SEQ ID NO: 24 - DNA, 20 bp, Artificial Sequence
  Description of Artificial Sequence Synthetic primer
cacgctgaagggaaatgtgg

SEQ ID NO: 25 - DNA, 21 bp, Artificial Sequence
  Description of Artificial Sequence Synthetic primer
gcaggactctgagcaaggatg

SEQ ID NO: 26 - DNA, 21 bp, Artificial Sequence
  Description of Artificial Sequence Synthetic primer
ggcgaagtacatgaggctgat

SEQ ID NO: 27 - DNA, 21 bp, Artificial Sequence
  Description of Artificial Sequence Synthetic primer
ggattgcctttcagttcacgc

SEQ ID NO: 28 - DNA, 22 bp, Artificial Sequence
  Description of Artificial Sequence Synthetic primer
aggtagaaggtgttcagctctg

SEQ ID NO: 29 - DNA, 22 bp, Artificial Sequence
  Description of Artificial Sequence Synthetic primer
ttcttgcgatacactctggtgc

SEQ ID NO: 30 - DNA, 22 bp, Artificial Sequence
  Description of Artificial Sequence Synthetic primer
cgggattgaatgttcttgtcgt

SEQ ID NO: 31 - DNA, 21 bp, Artificial Sequence
  Description of Artificial Sequence Synthetic primer
catgcaccgatgggatttcct

SEQ ID NO: 32 - DNA, 22 bp, Artificial Sequence
  Description of Artificial Sequence Synthetic primer
cgctaaagttagtgtctgtgct

SEQ ID NO: 33 - DNA, 49 bp, Artificial Sequence
  Description of Artificial Sequence Synthetic primer
caccgccatgtcccgacacactaactcgagttagtgtgtcgggacatgg

SEQ ID NO: 34 - DNA, 49 bp, Artificial Sequence
  Description of Artificial Sequence Synthetic primer
aaaaccatgtcccgacacactaactcgagttagtgtgtcgggacatggc

SEQ ID NO: 35 - DNA, 52 bp, Artificial Sequence
  Description of Artificial Sequence Synthetic primer
caccgccgtttgtaattctgcttatctcgagataagcagaattacaaacggc

SEQ ID NO: 36 - DNA, 53 bp, Artificial Sequence
  Description of Artificial Sequence Synthetic primer
aaaaagccgtttgtaattctgcttatctcgagataagcagaattacaaacggc

SEQ ID NO: 37 - DNA, 20 bp, Artificial Sequence
  Description of Artificial Sequence Synthetic primer
gccatggagaacgcgcacac

SEQ ID NO: 38 - DNA, 26 bp, Artificial Sequence
  Description of Artificial Sequence Synthetic primer
agaccagaacatatcgctaaagttag